(19) United States
(12) Patent Application Publication
(10) Pub. No.: US 2010/0226597 A1
(43) Pub. Date: Sep. 9, 2010

(54) BORTEZOMIB AND PROCESS FOR PRODUCING SAME

(75) Inventors: Raghavendracharyulu Venkata Palle, Hyderabad (IN); Rajasekhar Kadaboina, Hyderabad (IN); Veerendeer Murki, Hyderabad (IN); Amarendhar Manda, Hyderabad (IN); Nageshwar Gunda, Mahaboobnagar (IN); Ramaseshagiri Rao Pulla, Eluru (IN); Mallesha Hanmanthu, Nalgonda (IN); Narasimha Naidu Mopidevi, Hyderabad (IN); Suresh Kumar Ramdoss, Ramnad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LIMITED, Hyderabad 500 016, Andhra Pradesh (IN); DR. REDDY'S LABORATORIES, INC., Bridgewater, NJ (US)

(21) Appl. No.: 12/677,872

(22) PCT Filed: Sep. 12, 2008
(86) PCT No.: PCT/US2008/076178

§ 371 Date: Mar. 12, 2010

Related U.S. Application Data
(60) Provisional application No. 61/059,318, filed on Jun. 6, 2008.

(30) Foreign Application Priority Data
Sep. 12, 2007 (IN) 2053/CHE/2007
Jul. 24, 2008 (IN) 1784/CHE/2008

(51) Int. Cl. B65D 30/08; C07F 5/02; C07D 241/18

(52) U.S. Cl. 383/113; 544/229; 568/6; 544/406

(57) ABSTRACT

The present application provides a process for the preparation of Bortezomib, its intermediates and process for crystalline forms of Bortezomib.

### FIELD OF THE APPLICATION

The present application relates to processes for the preparation of Bortezomib and intermediate compounds useful for its preparation.

The present application also relates to process for the preparation of Bortezomib, which is substantially pure.

The present application further relates to processes for the preparation of crystalline forms A and B of Bortezomib. It also relates to the intermediate compounds and unique forms of Bortezomib.

### BACKGROUND OF THE APPLICATION

Bortezomib is the adopted name for the drug compound having the chemical name [(1R)-3-methyl-1-[[(2S)-1-oxo-3-phenyl-2-[(pyrazinyl carbonyl)amino]propyl]amino]butyl]boronic acid and is represented by the structural Formula I.

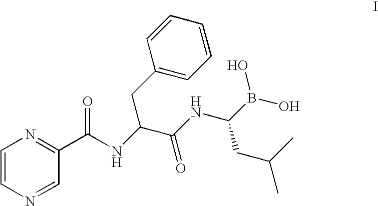

Bortezomib is an anti-neoplastic agent and is therapeutic proteosome inhibitor available in the market under the brand name “VELCADE®” in the form of injection. Each vial contains 3.5 mg of Bortezomib as a sterile lyophilized powder. In the US it is approved for the treatment of multiple myeloma and mantle cell lymphoma.

It was disclosed in the Chemistry review(s) section of Summary Basis Of Approval for Bortezomib (NDA 21-602) that the drug substance, drug product and the reconstituted drug product have three different molecular forms. PS-341 (Bortezomib) drug substance exists as the trimeric boroxine in the solid state. When exposed to water, the boroxine hydrolyses to monomeric boronic acid PS-341. The structure of the lyophilized PS-341 drug product has been determined to be symmetrical mannitol ester. While reconstituted by 0.9% NaCl solution, the reconstituted PS-341 drug product consists of equilibrium between the mannitol ester and the PS-341 boronic acid.

U.S. Pat. No. 5,780,454 discloses Bortezomib, its pharmaceutically acceptable salts, pharmaceutical composition and use to inhibit the proteosome function in a mammal. Further, it discloses a process for the preparation of Bortezomib and its analogues.

U.S. Pat. No. 6,713,446 discloses lyophilized formulation of Bortezomib esters. According to this patent, Bortezomib prepared by the process as described in U.S. Pat. No. 5,780,454 is white amorphous powder.

U.S. Pat. No. 4,525,309 discloses a process for the homologation of boronic esters by rearrangement of the intermediate boron “ate” complex in the presence of a Lewis acid catalyst to promote the rearrangement reaction and to minimize epimerization of alpha-carbon atom.

US 2005/0240047 A1 discloses processes for preparing Bortezomib intermediate, which are boronic ester compounds and Bortezomib itself.

The US '047 application discloses that the previously reported processes for the preparation of the intermediate compound of the formula-III

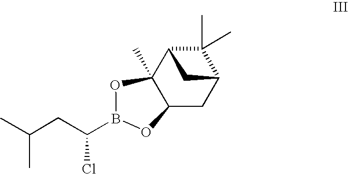

by Lewis acid promoted rearrangement of boron “ate” complex of the formula —X

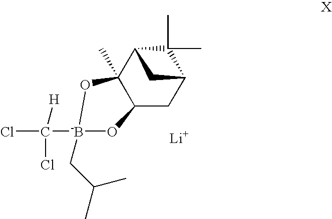

employ tetrahydrofuran, an ether solvent that is miscible with water, and requires rigorously dried equipment, solvents, and Lewis acid reagent and such reactions are expensive and difficult to scale up. Further, according to the '047 application, attempted scale-up of the prior art processes frequently results in further deterioration in diastereomeric ratio of the boronic ester compound either because of exposure of the product to halide ion during concentration of the reaction mixture to remove the tetrahydrofuran solvent and exchange it for a water-immiscible solvent or failure to completely remove the tetrahydrofuran during the subsequent aqueous washes.

The US '047 application appears to address the problems of the prior art by carrying out the rearrangement of the boron “ate” complex in an ether solvent that has low miscibility with water and a coordinating co-solvent. Non-limiting examples of low water miscible ether solvents identified in the '047 application for use in the process include tert-butyl methyl ether, tert-butyl ethyl ether, tert-amyl methyl ether, and isopropyl ether.

Further, the US '047 application discloses a process for the preparation of Bortezomib which comprises:
(i) Providing a biphasic mixture comprising the intermediate boronic ester compound of formula-IX,

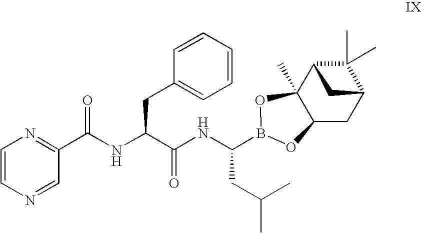

an organic boronic acid acceptor, a lower alkanol, a C₅₋₈ hydrocarbon solvent, and aqueous mineral acid;
(ii) stirring the biphasic mixture to afford Bortezomib;
(iii) separating the solvent layers; and
(iv) extracting Bortezomib or a boronic acid anhydride thereof into an organic solvent.

To enhance the purity of the product, the aqueous layer obtained after step (iii) is washed to remove neutral organic impurities prior to the extracting step (iv). Such process comprises the following steps:

1) separating the solvent layers;

2) adjusting the aqueous layer to basic pH;

3) washing the aqueous layer with an organic solvent; and

4) adjusting the aqueous layer to a pH of less than about 6.

Thus, the process described in the US '047 application comprises multiple organic solvent washings under acidic and basic conditions, followed by extracting the compound into an organic solvent, isolating the product and further recrystallization to obtain Bortezomib of enhanced purity.

It has been found that exposure of Bortezomib to an aqueous basic solution decrease the purity of Bortezomib. Particularly, when such process is performed on a large scale, exposure of Bortezomib to aqueous basic conditions for longer hours is difficult to avoid and hence this process may not be amenable for use on an industrial scale.

WO 2008/075376 A1 discloses crystalline forms I and II of Bortezomib and process for their preparation. Form-I of Bortezomib is prepared by using solvents such as acetone, CHCl₃, CH₂Cl₂ or nitriles and diluents such as Diisopropyl ether, Tertiary butyl methyl ether, n-hexane and n-heptane. Form-II of Bortezomib is prepared from hot solution of ethyl acetate. The application also discloses that, form-I and form-II are interconvertible by using the above described solvents.

There still exists a need to provide a simple and convenient process for the preparation of Bortezomib and its Intermediates.

### SUMMARY OF THE INVENTION

According to the present application, there are provided processes for the preparation of intermediates of Bortezomib and process for the preparation of Bortezomib, as well as the intermediates and Bortezomib produced thereby.

Further, the present application also provides processes for the preparation of Bortezomib, which is substantially pure and the substantially pure Bortezomib.

In one aspect, the present application provides a process for the preparation of intermediate compound of formula III

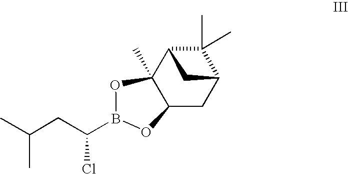

the process comprises the rearrangement of a boron “ate” complex of formula-X

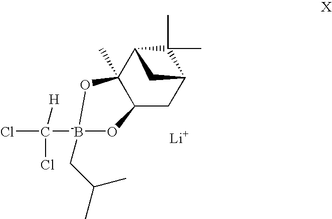

in the presence of Lewis acid catalyst, a water miscible ether solvent and excess dichloromethane. It has been found that the use of excess dichloromethane in the process for preparing the intermediate compound of formula-III, not only participates as reactant during the boron “ate” complex formation, but also assists in organic layer separation after quenching of the reaction mixture with aqueous acid solution. The intermediate compound of formula III obtained by the process of the present invention is subsequently utilized for the preparation of intermediate compound of formula-V

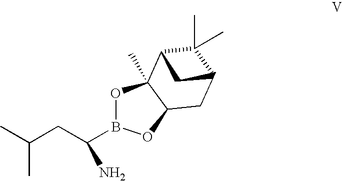

in the form of free base or acid addition salt form by the processes known in the prior art. The compound of formula-V can be subsequently utilized in the process for preparation Bortezomib by condensation reaction with intermediate compound of formula-VIII. The process for preparing intermediate compound of formula-VIII and intermediate-VIII are specific embodiments of the present application.

In an embodiment, the present application provides process for the preparation of intermediate compound of formula VIII

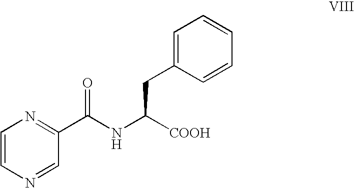

or salt which comprises the reaction of pyrazine carboxylic acid with L-phenylalanine in the presence of a condensing agent. Examples of condensing agents used in the process of the present application are selected from alkyl/aryl chloroformate, (1-Ethyl-3-(3-dimethylaminopropyl)carbodiimide/HOBt and DCC/N-hydroxy succinimide.

In one aspect, the present application provides process for the preparation of Bortezomib, which comprises:

(i) condensation of compound of formula V in the form of free base or acid addition salt with the compound of formula VIII or salt to produce (N-[(1S)-2-[[(1R)-1-[(3aS,4S,6S,7aR)-hexahydro-3a,5,5-trimethyl-4,6-methano-1,3,2-benzodioxaborol-2-yl]-3-methylbutylamino]-2-oxo-1-(phenylmethyl)ethyl]pyrazinecarboxamide (compound of Formula IX),

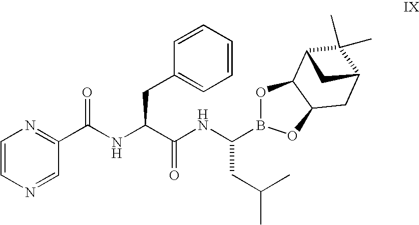

and
(ii) conversion of compound of formula IX to Bortezomib.

In another aspect, the present application provides a process for the preparation of Bortezomib, the process comprising:
a) reacting (N-[(1S)-2-[[(1R)-1-[(3aS,4S,6S,7aR)-hexahydro-3a,5,5-trimethyl-4,6-methano-1,3,2-benzodioxaborol-2-yl]-3-methylbutylamino]-2-oxo-1-(phenylmethyl)ethyl]pyrazinecarboxamide (compound of Formula IX)

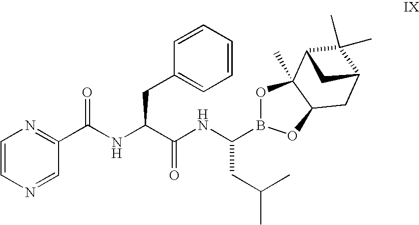

with an organic boronic acid acceptor and aqueous mineral acid in the presence of an alcohol solvent and an aliphatic hydrocarbon solvent;
b) separating the aqueous layer;
c) extracting the aqueous layer with a water immiscible organic solvent, which is other than aliphatic hydrocarbon solvent; and
d) isolating Bortezomib.

In another aspect, the present application provides a process for the purification of Bortezomib comprising:
a) providing a solution of Bortezomib in an organic solvent;
b) precipitating the product by adding an anti-solvent; and
c) separating of the obtained product.

The process of the present application provides, in some embodiments, substantially pure Bortezomib free of its stereo isomers and/or impurities and having a purity of greater than about 95% by HPLC.

Forms A and B discussed herein, and indeed any form of Bortezomib, may be purified using the solvents described herein including, without limitation, methanol, water, ethylacetate, toluene and dichloromethane. Thus, solvents that can be used to produce Form A may be used to purify Form B and solvents used to produce Form B may be used to purify Form A.

Further, the present application also provides processes for the preparation of crystalline forms A and B of Bortezomib.

It was surprisingly discovered that Form A solid obtained by the process of the present application is a monomer rather than the trimeric anhydride. Form A is another aspect of the invention. Form A can be produced using a solvent system of methanol and water.

In another embodiment, the present application provides a process for the preparation of crystalline Form A of Bortezomib, comprising:
a) providing a solution of Bortezomib in an alcohol, and in particular, methanol;
b) adding water to precipitate the solid; and
c) isolating the obtained solid.

In yet another embodiment, the present application provides a process for the preparation of crystalline Form B of Bortezomib, comprising:
a) providing a solution of Bortezomib in a halogenated alkane solvent or an ester solvent
b) adding an aromatic hydrocarbon solvent to precipitate the solid; and
c) isolating the obtained solid. Form B is still another embodiment of the invention. In one embodiment, Form B is produced using a solvent system of one of either ethyl acetate or dichloromethane mixed with toluene.OK

In another embodiment, the present application provides a pharmaceutical composition containing a pharmaceutically effective amount of Bortezomib obtained by the processes of present application and at least one pharmaceutically acceptable excipient.

The present application also provides a storage system for stabilizing Bortezomib. The storage system of the present application preferably comprises at least one sealed polymeric bag, (e.g., a transparent or opaque polyethylene bag-having thickness of about 0.10 mm to about 0.50 mm) or a combination of such bags, which, if desired, may be sealed inside of a laminated aluminum bag. Optionally, an oxygen absorbent and a moisture absorbent (or desiccant) may be included between one or more of such bags. Finally, the packed samples are stored in HDPE containers.

The defined drug packaging system of the present application may prevent the degradation of Bortezomib over long storage periods. Preferably, the storage system is capable of reducing or eliminating drug instability due to possible contact with air and/or water in the atmosphere.

### BRIEF DESCRIPTION OF THE DRAWINGS

FIG. 1: Illustrative example of X-ray powder diffraction (XRPD) pattern of Bortezomib Form A prepared according to Example 8.

FIG. 2: Illustrative example of X-ray powder diffraction (XRPD) pattern of Bortezomib Form A prepared according to Example 9.

FIG. 3: Illustrative example of differential scanning calorimetry (“DSC”) curve of Bortezomib Form A prepared according to Example 9.

FIG. 4: Illustrative example of thermogravimetric analysis (TGA) curve of Bortezomib Form A prepared according to Example 9.

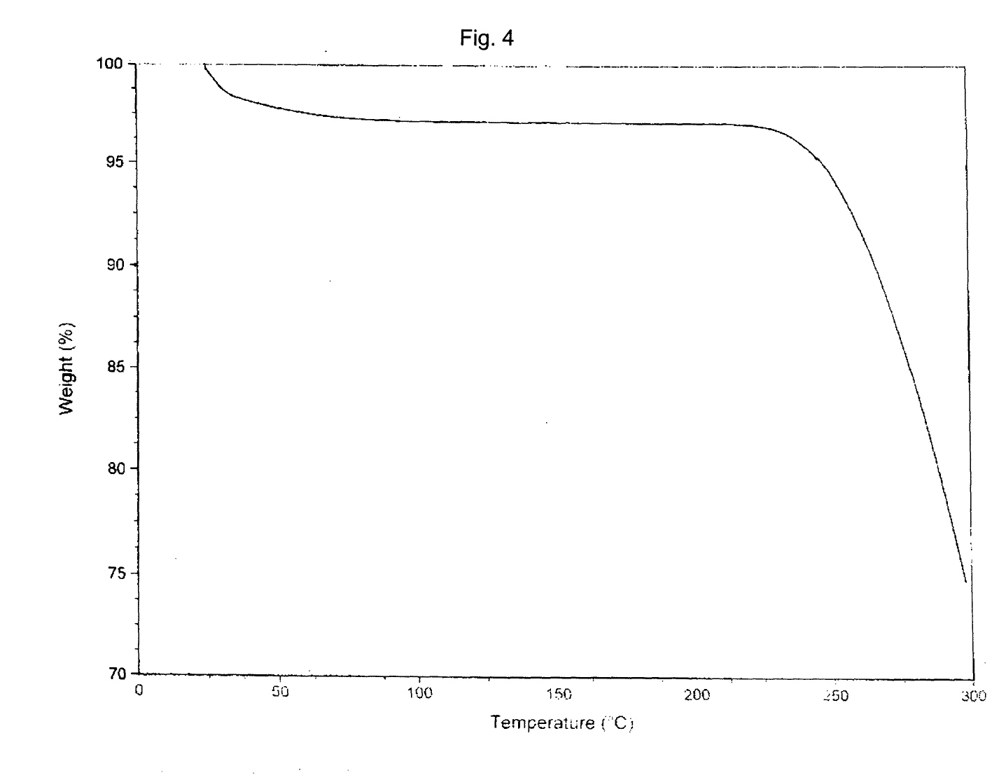

FIG. 5: Illustrative example of X-ray powder diffraction (XRPD) pattern of Bortezomib Form-B prepared according to Example 2.

FIG. 6: Illustrative example of thermogravimetric analysis (TGA) curve of Bortezomib Form-B prepared according to Example 2.

FIG. 7: Illustrative example of infrared absorption spectrum of Bortezomib Form-B prepared according to Example 2.

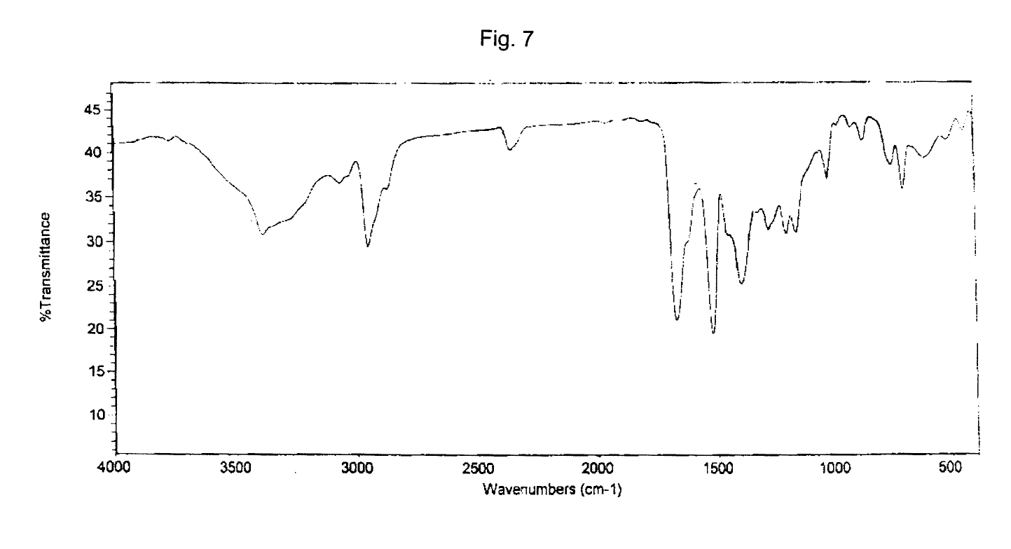

FIG. 8: Illustrative example of example of X-ray powder diffraction (XRPD) pattern of Bortezomib prepared according to Example 6 which is Form B. OK

FIG. 9: Illustrative example of thermogravimetric analysis (TGA) curve of Bortezomib prepared according to Example 6.

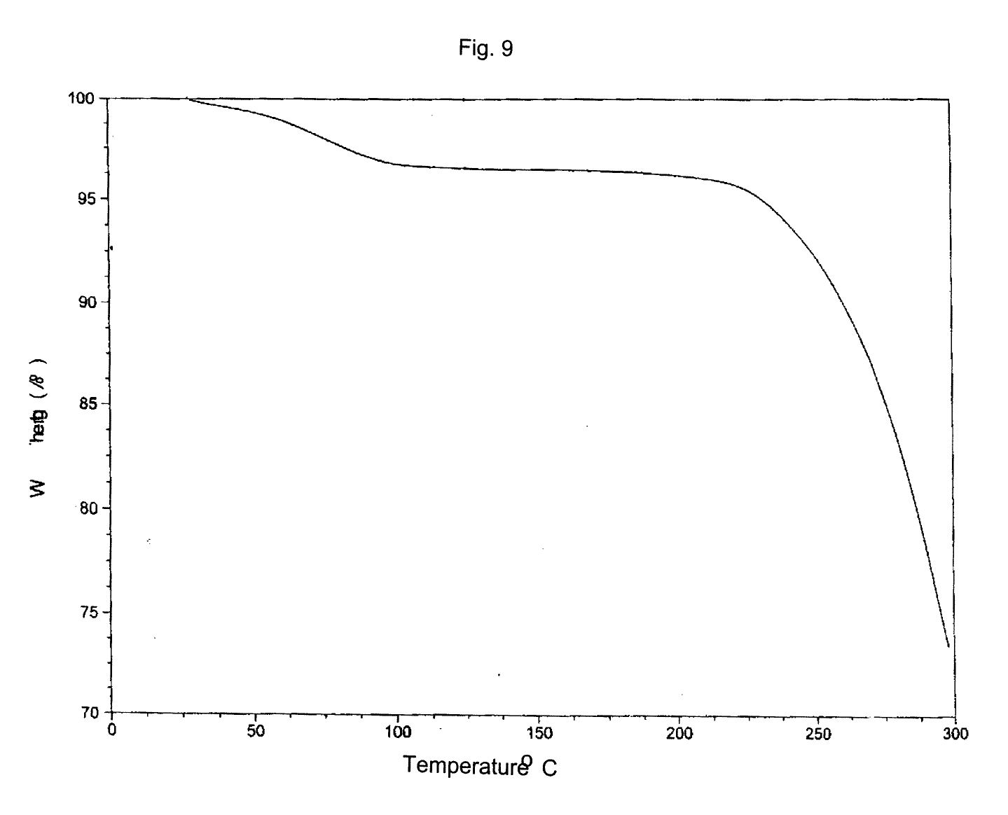

FIG. 10: Illustrative example of infrared absorption spectrum of Bortezomib prepared according to Example 6.

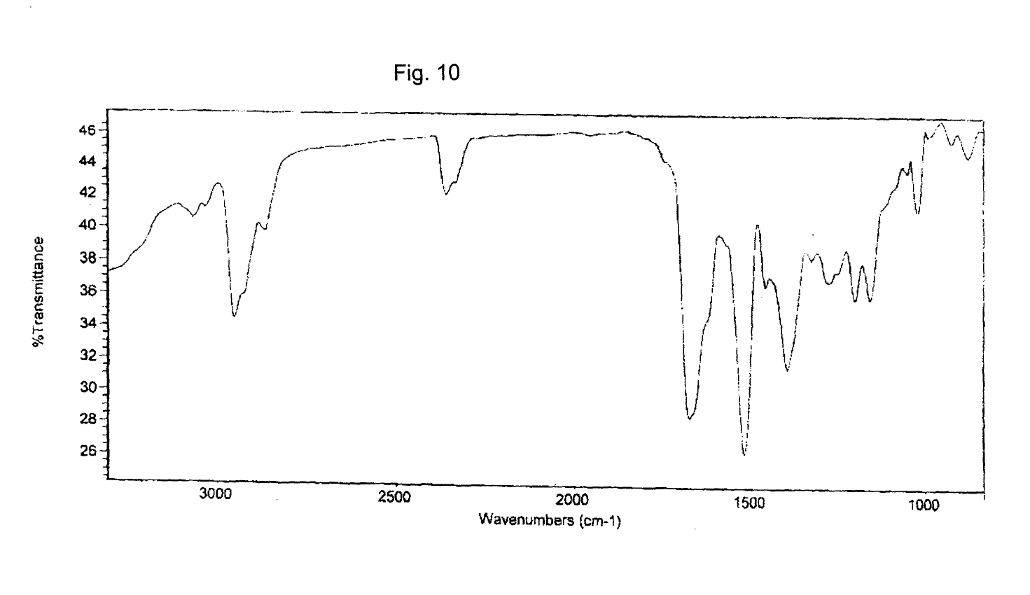

### DETAILED DESCRIPTION OF THE APPLICATION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values.

Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Note that while the specification and claims may refer to a final product such as, for example, a tablet or other dosage form of the invention as, for example, containing particles having a certain particle size or distribution, or a certain type of, for example, a specific form of a filler, it may be difficult to tell from the final dosage form that the recitation is satisfied. However, such a recitation may be satisfied if the materials used prior to final production (in the case of a tablet for example, blending and tablet formulation), for example, meet that recitation. Indeed, as to any property or characteristic of a final product which cannot be ascertained from the dosage form directly, it is sufficient if that property resides in the components recited just prior to final production steps.

Where this document refers to a material, such as in this instance, Bortezomib, and the unique crystalline forms, salts, solvates and/or optical isomers thereof by reference to patterns, spectra or other graphical data, it may do so by qualifying that they are “substantially” shown or depicted in a Figure, or by one or more data points. By “substantially” used in such a context, it will be appreciated that patterns, spectra and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art should be able to compare the figures herein with a pattern generated of an unknown form of, in this case, Bortezomib, and confirm its identity as one of the forms disclosed and claimed herein. The same holds true for other techniques which may be reported herein.

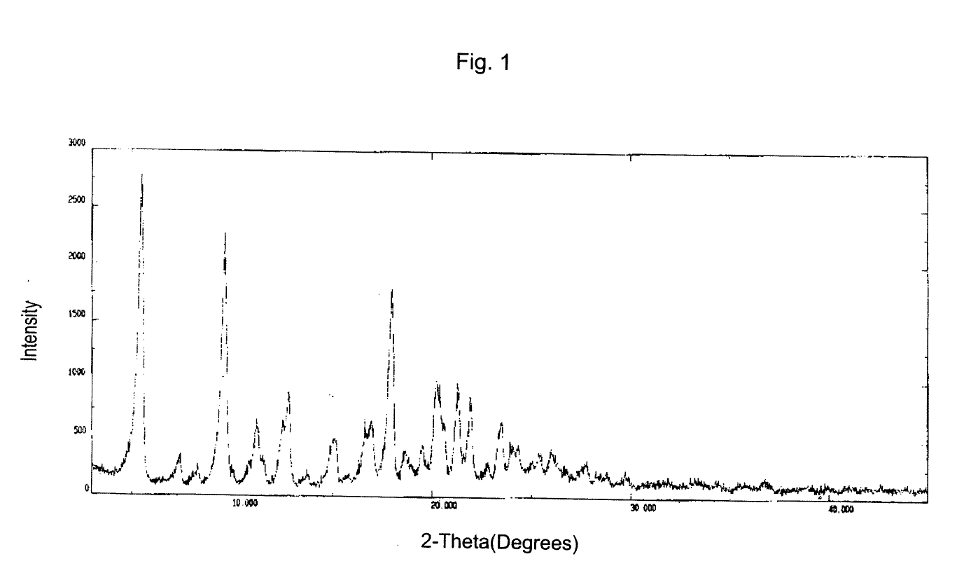

In addition, where a reference is made to a figure, it is permissible to, and this document includes and contemplates, the selection of any number of data points illustrated in the figure which uniquely define that crystalline form, salt, solvate, and/or optical isomer, within any associated and recited margin of error, for purposes of identification. Again, and for example, for a crystalline form of Bortezomib. It is permissible to select any number of PXRD peaks represented in FIG. 1, often between 4 and 10, which +/−0.2 degrees two theta, uniquely identify that form, as a way of describing and claiming that material.

A reference to a molecule such as, in this case, Bortezomib, unless otherwise specified or inconsistent with the disclosure in general, refers to any salt, crystalline or amorphous form, optical isomer and/or solvate form thereof.

When a molecule or other material is identified herein as “pure”, it generally means, unless specified otherwise, that the material is about 99% pure or more. In general, this refers to purity with regard to unwanted residual solvents, reaction byproducts, impurities and unreacted starting materials. In the case of stereoisomers or polymorphs, “pure” also means 99% of one enantiomer or diastereomer or polymorph, as appropriate.

The term “Substantially pure Bortezomib” as used herein shall be understood to mean Bortezomib having a purity of more than about 95% by HPLC with little to no content of undesired stereo isomers and/or other impurities. The amount of any undesired stereo isomer or other impurity in Bortezomib, if present, will be in relatively minor amounts, e.g., less than about 5, preferably less than about 1, more preferably less than about 0.5, most preferably less than about 0.2 weight percent by HPLC based on the weight of Bortezomib.

The present application provides processes for the preparation of intermediates of Bortezomib and process for the preparation of Bortezomib.

In one aspect, the present application provides a process for the preparation of an intermediate compound of formula III

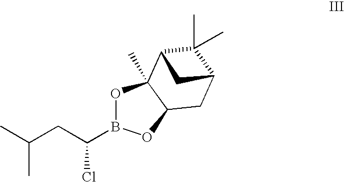

the said process comprises the rearrangement of boron “ate” complex of formula-X

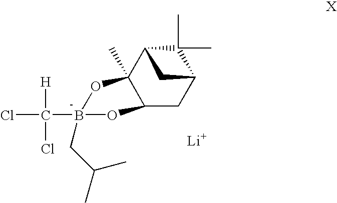

in the presence of Lewis acid catalyst, a water miscible ether solvent and dichloromethane.

In one embodiment, the process comprises the steps of:

I. Formation of compound of formula X by adding lithium diisopropyl amide (LDA) to a solution of the compound of formula-II

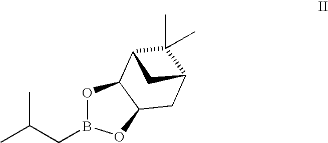

in a solvent mixture comprising dichloromethane and water miscible ether solvent followed by maintaining the resulting solution at a temperature of about −40 to −70° C. for about 10 to about 60 minutes and in one embodiment, about 30 minutes;
II. Adding a mixture of zinc chloride in tetrahydrofuran into the product of step I followed by maintaining the reaction mass at a temperature of about −40 to −70° C. often for about 30 to about 120 minutes and in one embodiment, about 60 minutes;
III. Raising the reaction temperature to from about 10° C. to about ambient temperature (25° C.);
IV. Adding an aqueous acid solution; and
V. Optionally separating the organic layer containing the compound of formula-III.

This can be followed, if desired, by washing the organic layer with brine and/or concentrating the organic layer to isolate the compound of formula-III.

Use of excess dichloromethane in the process for preparing the intermediate compound of formula-III is a preferred embodiment of the above process. Without wishing to bound by the theory, it is believed that use of excess dichloromethane in the process for preparing the intermediate compound of formula-III, which is a specific aspect of the present application, not only participates as reactant during the boron “ate” complex formation, but also assists in organic layer separation after quenching of the reaction mixture with aqueous acid solution. The specifics of a preferred embodiment of this process are detailed in step-b of Example 1.

In one embodiment, the dichloromethane may be utilized in the range of about 4 moles to about 8 moles per mole of the compound of formula-II. This range may also be between about 5 moles to about 6 moles per mole of the compound of formula-II.

Water miscible ether solvents used in the process of the present application include tetrahydrofuran, which may be utilized in the range of about 10 to about 20 times per gram of compound of formula-II. This range may also be between about 15 to about 17 times and in another embodiment, about 16 times to that of compound of formula-II.

The amount of zinc chloride that may be utilized in the reaction may be molar excess compared with that of the compound of formula II. It may be present in an amount of about 1.2 to about 2.0 moles per mole of the compound of formula II. It may be used in an amount of about 1.7 to about 1.8 moles per mole of the compound of formula II. Commercially available zinc chloride having moisture content up to about 6% w/w can be used in the process of the present application without affecting the intended result.

n-Hexyl lithium and Diisopropyl amine utilized for preparing the LDA mixture may be used in an amount of from about 1 mole to about 1.5 moles, individually, with respect to the compound of formula-II. They may also individually be used in an amount of from about 1.2 moles to about 1.3 moles per mole of the compound of formula II. n-Hexyl lithium and Diisopropyl amine are used in molar proportions with respect to each other.

The acid that may be utilized for quenching the reaction mass may be either organic acid or inorganic acid. The inorganic acids may be selected from hydrochloric acid, sulphuric acid or phosphoric acid. Preferably, the organic acid may be selected from tartaric acid, citric acid.

The strength of the aqueous acid solution used for quenching the reaction mass may range from about 5% to about 20% w/w. In another embodiment, the strength is about 10% to about 12% w/w of acid solution. The pH of the reaction mixture before separation of the organic and aqueous layers may be between about 0.5 to about 3.

The intermediate compound of formula III obtained by the process of the present application is subsequently utilized for the preparation of intermediate compound of formula-V

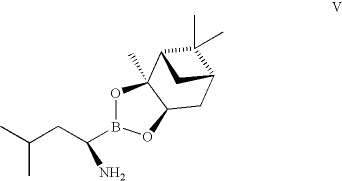

in the form of free base or acid addition salt form by the processes similar to known in the prior art (The Journal Of Biological Chemistry; Vol 259, No. 24, pp 15106-15114, Dec. 25, 1984; U.S. Pat. No. 7,223,745) as delineated in the Scheme-1 herein below:

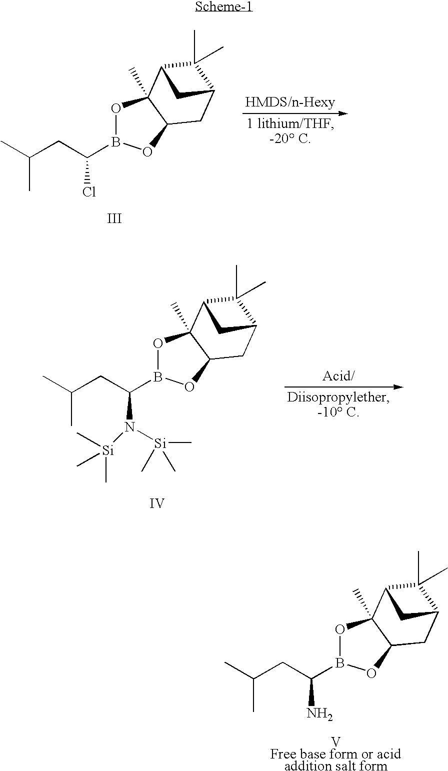

The compound of formula V used in the process of the present application may be in the form of trifluoroacetic acid salt.

The specific details for the step of converting intermediate compound of formula-III into compound of formula-IV and its subsequent conversion into compound of formula V are provided in the steps c and d of the Example-1.

Since the compound of formula V in the form of free base or acid addition salt form is the key starting material for the preparation of Bortezomib, it is desirable to have compound of formula V with improved purity as measured by gas chromatography (GC) that would not affect the yield and purity of Bortezomib. The compound of formula V in the form of free base or acid addition salt form obtained by the process of the present application may, in some embodiments, have a purity greater than about 95%, preferably greater than about 98% and more preferably greater than about 99.5% as measured by GC.

The intermediate compound of formula-V in the form of free base or acid addition salt form can be subsequently utilized in the process of the present application for preparation Bortezomib by condensation with intermediate compound of formula-VIII. The process for preparing intermediate compound of formula-VIII is one of the specific embodiments of the present application.

In an embodiment, the present application provides a process for the preparation of intermediate compound of formula VIII

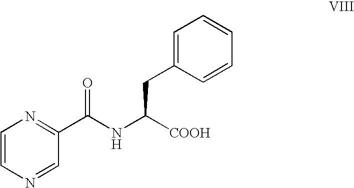

which comprises the reaction of pyrazine 2-carboxylic acid with L-phenylalanine or salt in the presence of alkyl/aryl chloroformate as depicted in the scheme 2 given below.

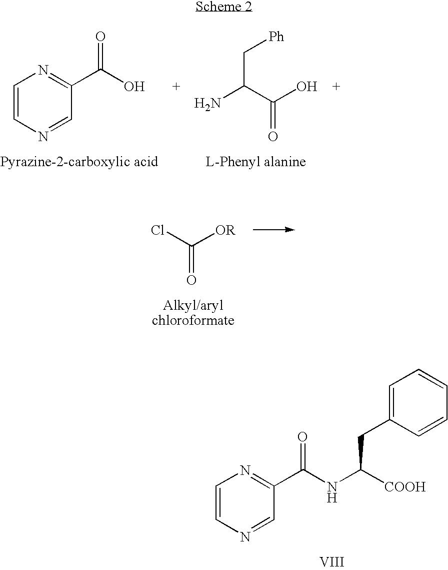

wherein R represents an optionally substituted alkyl/aryl group.

The alkyl/aryl chloroformate that may be utilized for the preparation of compound of formula VIII includes, but are not limited to ethylchloroformate, benzylchloroformate, para nitrophenylchloroformate.

The coupling reaction may preferably be carried out in a ketone solvent in the presence of base at a temperature in the range of about −20° C. to about 40° C. The ketone solvent may be selected from acetone, methyl isobutyl ketone, ethyl methyl ketone, and the like. Water may be present as co-solvent for the reaction.

The base used in the condensation reaction includes, but is not limited to, inorganic bases such as sodium hydroxide, potassium hydroxide, and the like; organic bases such as alkyl amines which include triethyl amine, diisopropylethylamine, pyridine, dimethylaminopyridine, diazabicycloundecane, N-methyl morpholine and the like. Mixtures of any of the organic and/or inorganic bases specified above may also be used for the said reaction.

An embodiment of the present application also provides an alternate process for preparing N-(2-pyrazinecarbonyl)-L-phenylalanine of formula VIII as represented in scheme 3,

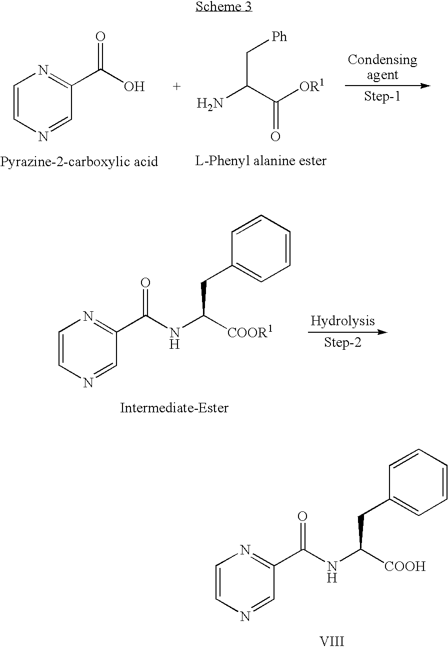

wherein R′ represents an optionally substituted alkyl group.

The optionally substituted alkyl group includes but is not limited to methyl, ethyl, propyl, tert-butyl, optionally substituted benzyl.

The process comprises two steps involving:

(i) condensation of pyrazine-2-carboxylic acid with optionally substituted alkyl ester of L-phenylalanine or its salt in the presence of condensing agent, in one embodiment, in the presence of a base; and

(ii) hydrolysis of the ester functional group obtained in the product of step (i), in one embodiment, using aqueous alkali solution.

The amount of substituted pyrazine 2-carboxylic acid used in step (i) may range from about 1.0 mole to about 1.8 molar equivalents per mole of alkyl ester of L-phenylalanine or its salt. 1.2 moles per mole of alkyl ester of L-phenylalanine or its salt may also be used.

Condensing agents that can be used in step (i) may be selected from the combinations dicyclohexylcarbodiimide/N-hydroxysuccinimide and Ethyl-3-(3-dimethylaminopropyl) carbodiimide or salt thereof /N-hydroxybenzotriazle.

The amount of dicyclohexylcarbodiimide/N-hydroxysuccinimide and Ethyl-3-(3-dimethylaminopropyl) carbodiimide or salt thereof /N-hydroxybenzotriazle used individually may range from about 1.0 mole to about 1.8 moles per mole of alkyl ester of L-phenylalanine or its salt. 1.2 moles may be used. N-hydroxysuccinimide and N-hydroxybenzotriazle are often used in equimolar proportions with respect to dicyclohexylcarbodiimide and Ethyl-3-(3-dimethylaminopropyl) carbodiimide or salt thereof respectively.

The base used in the condensation reaction may include, but is not limited to diisopropylethylamine, pyridine, dimethylaminopyridine, diazabicycloundecane, N-methyl morpholine. The amount of base used may range from about 1.0 mole to about 2.0 moles per mole of alkyl ester of L-phenylalanine or its salt. Preferably, 1.5 moles of the base may also be used.

The condensation reaction of step (i) may be carried out in solvents like DMF, DMA, or ketone solvents that may be selected from acetone, methyl isobutyl ketone, ethyl methyl ketone, and the like.

The temperature at which the reaction may be carried out may range from about −20° C. to about 60° C. The reaction may be carried out at a temperature of about 0 to about 30° C.

The reaction may be carried out for a suitable period of time. If desired, the product obtained (intermediate ester) from step (i) may be isolated before hydrolysis by general workup procedures or by process as disclosed in the present application.

The hydrolysis of the ester functional group of the product obtained in step (i) may be carried out preferably using an aqueous alkali solution. Optionally organic solvent may also present as a co-solvent for the hydrolysis step.

Suitable bases that can be used for the hydrolysis may include but are not limited to sodium hydroxide, potassium hydroxide, and the like.

The amount of base used for ester hydrolysis may be determined by a person ordinary skilled in the art. For instance, if the intermediate ester of product of step (i) is isolated before hydrolysis, the amount of based used for hydrolysis may range from about 1.0 mole to about 2.0 moles per mole of the isolated ester of product of step (i). 1.1 moles of the base may also be used.

The organic solvent used as a co-solvent for hydrolysis reaction of step (ii) may be selected from solvents—acetone, methyl isobutyl ketone, ethyl methyl ketone, methanol, ethanol, isopropanol.

The temperature at which the reaction may be carried out may range from about 0° C. to about 60° C. The reaction may also be carried out at a temperature of about 25° C. to about 35° C. The reaction may be carried out for a suitable period of time, and the product obtained (compound of formula VIII) may be isolated by general workup procedures or by process as disclosed in the present application.

In another aspect, the present invention provides the compound N-(2-pyrazinecarbonyl)-L-phenylalanine of formula VIII

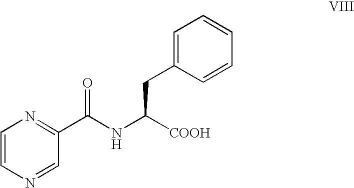

which is an intermediate in the preparation of Bortezomib, having purity equal to or greater than 95% by HPLC.

Since the compound of formula VIII is the key starting material for the preparation of Bortezomib, it is desirable to have compound of formula VIII with both chemical and chiral HPLC purity that would not affect the yield and purity of Bortezomib. The compound of formula VIII obtained by the process of the present application has both chemical and chiral HPLC purity greater than about 95%, preferably greater than about 99%, more preferably greater than about 99.5% and most preferably greater than about 99.8%.

Condensation of the compound of formula VIII i.e. N-(2-pyrazinecarbonyl)-L-phenylalanine or salt with the compound of formula V in the form of free base or acid addition salt form by any of the known methods to produce the compound of formula X and subsequent conversion of compound of formula IX into Bortezomib is another aspect of the application as represented herein below in scheme 4.

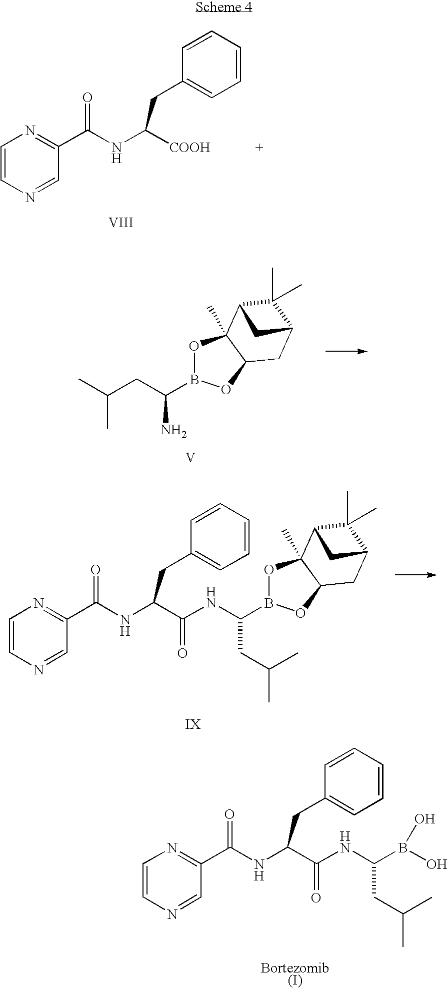

The specific details for the condensation reaction of compound of formula VIII or salt and compound of formula V in the form of free base or acid addition salt form are provided in the step (h) of Example-1.

In a further aspect, the present application provides a process for the preparation of Bortezomib, said process comprising:
a) reacting (N-[(1S)-2-[[(1R)-1-[(3aS,4S,6S,7aR)-hexahydro-3a,5,5-trimethyl4,6-methano-1,3,2-benzodioxaborol-2-yl]-3-methyl butylamino]-2-oxo-1-(phenylmethyl)ethyl]Pyrazinecarboxamide (compound of Formula IX)

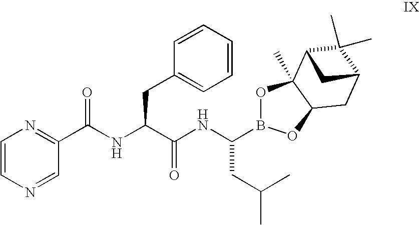

with organic boronic acid acceptor and aqueous mineral acid in the presence of an alcohol solvent and an aliphatic hydrocarbon solvent; and
b) separating the aqueous layers
c) extracting the aqueous layer with a water immiscible organic solvent, which is other than aliphatic hydrocarbon solvent; and optionally
d) isolating Bortezomib.

All the steps for process of preparation of Bortezomib from compound of formula IX according to the present application are independently described below. Step-a)

Step a) involves reaction of a compound of formula IX

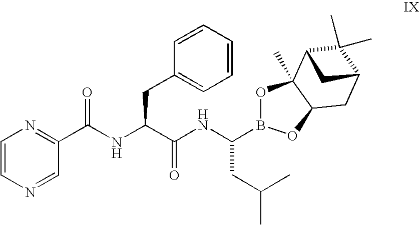

with an organic boronic acid acceptor and aqueous mineral acid in the presence of an alcohol solvent and an aliphatic hydrocarbon solvent to give compound of formula I.

The organic boronic acid acceptors that may be used in step-a) includes, but are not limited to, butyl boronic acid, isobutyl boronic acid, phenylboronic acid, benzyl boronic acid, and the like. In one embodiment, isobutyl boronic acid is used as the boronic acid acceptor.

The amount of organic boronic acid acceptor used in step a) may range from about 1 mole to about 1.5 molar equivalents, per mole of compound of Formula IX. 1.2 moles per mole of compound of Formula IX may be used.

The mineral acid used in the reaction may be selected from hydrochloric acid, sulphuric acid, phosphoric acid. In one embodiment, hydrochloric acid is used. The concentration of aqueous mineral acid used may range from about 0.5 N to about 3N. Aqueous mineral acid of 1N concentration may also be used. The quantity of aqueous mineral acid used for the reaction may vary from about 5-25 ml/gm of the compound of formula IX. The concentration and quantity of aqueous mineral acid used in the reaction can be readily determined by a person ordinarily skilled in the art.

The alcohol solvents that may be used in the process of step a) includes, but are not limited to, C₁-C₄ alcohols such as methanol, ethanol, Isopropanol, butanol or mixtures thereof. The aliphatic hydrocarbon solvents that may be used in the process of step a) includes, but are not limited to, O₅ to C₁₀ straight or branched alkanes or cycloalkanes such as n-pentane, n-hexane, n-heptane, cyclohexane or mixture thereof. In some embodiments, a solvent mixture comprising methanol and n-heptane may be used as the reaction solvent.

The process of step a) may be carried out at a temperature of from about 25° C. to about reflux temperature of the solvent used. Indeed, it may be carried out at a temperature of about 25° C. to 35° C.

### Step-b)

Step-b) comprises separation of the aqueous layer.

After completion of the reaction, the aqueous layer may be separated from the reaction mixture and the organic layer is discarded. The aqueous layer optionally may be washed, preferably, with a C₅-C₈ aliphatic hydrocarbon solvent such as n-heptane. The washing may be carried out by vigorous stirring of the aqueous layer with an aliphatic hydrocarbon solvent for about 10-15 minutes and separating the organic layer, which may be discarded. Optionally, the process may be repeated 1 to 3 more times.

The obtained aqueous layer after the optional washing step may be concentrated, with or without vacuum.

### Step-c)

Step c) comprises extracting the aqueous layer with a water immiscible organic solvent, which is other than aliphatic hydrocarbon solvent.

The aqueous layer obtained in step-b) may be extracted with a water immiscible organic solvent, which is other than aliphatic hydrocarbon solvent. The extraction process may be carried out by adding the solvent to the aqueous layer and vigorous stirring for 10-15 minutes followed by separating the organic layer.

The water immiscible organic solvent that may be used for extraction include, but are not limited to, alcoholic solvents such as isobutanol, and t-butanol; halogenated solvents such as dichloromethane, 1,2-dichloroethane and chloroform; ester solvents such as ethyl acetate, n-propyl acetate, isopropylacetate and n-butyl acetate; or mixtures thereof. The solubility of water in the organic solvent selected for extraction should be less than about 10% w/w, preferably less than about 2% w/w. In one embodiment, a halogenated alkane is used as the extracting solvent. In still another embodiment, dichloromethane is used as the extracting solvent.

The extraction process may be repeated till Bortezomib is completely extracted into the organic solvent. The organic layers obtained in different extractions are combined, and optionally washed with saturated sodium bicarbonate solution followed by brine solution and concentrated either completely or to a minimum volume under vacuum to give a residue or a concentrated solution of Bortezomib. The concentrated solution or residue may be optionally cooled to a temperature of 25° C. to 35° C.

### Step-d)

Step d), which is optional, involves isolation of the product.

The isolation of the product may be carried out by methods such as cooling, seeding, or adding an organic solvent to the concentrated solution or residue, or a combination thereof.

In one embodiment, the solid may be isolated by method such as adding an organic solvent to the concentrated solution or residue of step c).

Organic solvents that may be used for isolation include, but are not limited to, hydrocarbon solvents such as toluene, xylenes, cyclohexane, n-hexane, n-heptane; halohydrocarbon solvents such as dichloromethane, dichloroethane; ester solvents such as ethylacetate, propylacetate or mixtures thereof. In one embodiment, toluene may be used to isolate the product. A mixture of toluene with either a halohydrocarbon solvent or an ester solvent may also be used. However, if the mixture of solvents is used for isolation, the ratio of the individual solvents in the mixture may range from about 2 to 98% v/v.

The solvent may be added to the Bortezomib concentrated solution or residue obtained after step-c) for a period of sufficient time such as for about 15 minutes to 2 hours or more to affect precipitation. Suitable temperature may range from about 0° C. to about 50° C. The obtained reaction mixture may then be stirred for about 30 minutes to 5 hours, or longer hours to affect complete precipitation. The reaction mixture may be stirred at 25° C. to 35° C. for about 2 to about 3 hours.

The obtained precipitate may be separated by the techniques known in the art. One skilled in the art may appreciate that there are many ways to separate the solids from heterogeneous mixtures. For example, it may be separated by using any techniques such as filtration by gravity or by suction, centrifugation, decantation, and the like. After separation, the solid may optionally be washed with suitable solvent.

The solid thus obtained may be dried. Drying may be suitably carried out in a tray dryer, vacuum oven, air oven, fluidized bed drier, spin flash dryer, flash dryer and the like. The drying may be carried out at temperature of about 35° C. to about 70° C., and preferably at about 50° C., optionally under reduced pressure. The drying may be carried out for any time period necessary for obtaining the product with desired purity, such as from about 1 to about 25 hours, or longer.

In another aspect, the present application provides a process for the purification of Bortezomib comprising:

a) providing a solution of Bortezomib in an organic solvent,

b) precipitating the product by adding an anti-solvent;

c) separating the obtained product.

The process steps for purification of Bortezomib are separately described herein below:

### Step a)

### Step a) Involves Providing a Solution of Bortezomib in an Organic Solvent.

Providing a solution of Bortezomib in an organic solvent includes the solution of a chemical reaction by which Bortezomib is prepared or dissolution of Bortezomib in an organic solvent, optionally under nitrogen atmosphere. Any form of Bortezomib having purity of about 90% or more is acceptable for providing the solution. Any form of Bortezomib, such as amorphous or crystalline form or mixtures of amorphous and crystalline forms of Bortezomib in any proportions obtained by any method may be used for providing the solution.

Organic solvents that may be used for the dissolution includes, but are not limited to, alcoholic solvents such as methanol, ethanol, isopropyl alcohol, n-butanol, isobutanol, and t-butanol; halogenated solvents such as dichloromethane, 1,2-dichloroethane, chloroform; ester solvents such as ethyl acetate, n-propyl acetate, isopropylacetate and n-butyl acetate; nitrile solvents such as acetonitrile, propionitrile; or mixtures thereof. Preferably, Methanol, isopropyl alcohol, dichloromethane or ethylacetate may be used for the purification of Bortezomib.

Solution of Bortezomib may be provided at a temperature of about 20° C. to a temperature up to boiling point of the solvent used. Preferably, the solution is provided at a temperature of about 25° C. to about 35° C.

The undissolved particles may be removed suitably by filtration, centrifugation, decantation, and other techniques. Depending upon the equipment used, concentration and temperature of the solution, the filtration apparatus may need to be preheated to avoid premature crystallization.

### Step b)

Step b) involves precipitating the product by adding an anti-solvent:

The Bortezomib solution of step a) may be combined with an anti-solvent for precipitation. The addition of anti-solvent may be carried out over the period of about 5 minutes to about 1 hour or more. The temperature at which the anti-solvent may be added may range from about 0-45° C. The temperature used may be ambient temperature (up to 25° C.).

The resulting suspension is maintained at a temperature of about 0° C. to about 35° C. The obtained mixture is stirred for about 30 minutes to about 5 hours or more to affect the complete precipitation. In one embodiment, the suspension of Bortezomib is maintained at a temperature of about 25° C. to about 35° C. for 2 to 3 hours.

Anti-solvent that may be used in the process of the present invention include, but are not limited to, water, hydrocarbons such as toluene, xylene, cyclohexane, n-hexane, n-heptane; ethers such as diethyl ether, diisopropyl ether, tetrahydrofuran (THF), 1,4-dioxane, dimethoxyethane, methyl tertiary-butyl ether; or mixtures thereof. In a particular embodiment, either toluene or diisopropyl ether is used as the anti-solvent.

### Step c)

### Step c) Involves Separation of the Product.

The obtained precipitate may be separated by the techniques known in the art. One skilled in the art may appreciate that there are many ways to separate the solid from the mixture. For example, it may be separated by any techniques such as filtration by gravity or by suction, centrifugation, decantation, and the like. After separation, the solid may optionally be washed with suitable solvent.

The wet solid may be further dried. Drying may be suitably carried out in a tray dryer, vacuum oven, air oven, fluidized bed drier, spin flash dryer, flash dryer and the like. The drying may be carried out at temperatures of about 35° C. to about 70° C. and, in one embodiment, about 50° C., optionally under reduced pressure. The drying may be carried out for any time period necessary for obtaining the product with desired purity, such as from about 1 to about 40 hours, or longer.

The purification process may optionally be repeated till Bortezomib of desired purity is achieved. For example, purification may be continued until essentially pure, substantially pure or pure Bortezomib is obtained.

In another one embodiment, the present application provides process for the purification of Bortezomib, wherein the solvent is isopropyl alcohol and the anti-solvent is isopropyl ether.

In yet another embodiment, the present application provides a purification process for the preparation substantially pure Bortezomib comprising the following steps a)-c):
a) Providing a solution of Bortezomib in an organic solvent selected from alcohol, halogenated solvents, esters, nitriles, hydrocarbon, ether or mixtures thereof;
b) a step of adding, where necessary, an anti-solvent to the solution obtained in step a).
c) isolating the solid product from step-a) or step-b)
   The process steps for preparation substantially pure Bortezomib are separately described herein below:

### Step a) Involves Providing a Solution of Bortezomib in an Organic Solvent.

Organic solvents that may be used for the dissolution includes, but are not limited to, alcoholic solvents such as methanol, ethanol, isopropyl alcohol, n-butanol, isobutanol, and t-butanol; halogenated solvents such as dichloromethane, 1,2-dichloroethane, chloroform; ester solvents such as ethyl acetate, n-propyl acetate, isopropylacetate and n-butyl acetate; nitrile solvents such as acetonitrile, propionitrile; hydrocarbons such as toluene, xylene, cyclohexane, n-hexane, n-heptane; ethers such as diethyl ether, diisopropyl ether, tetrahydrofuran (THF), 1,4-dioxane, dimethoxyethane, methyl tertiary-butyl ether; or mixtures thereof.

Solution of Bortezomib may be provided by a process as described in the above embodiment of a process for the purification of Bortezomib.

Step b) Involves a Step of Adding, where Necessary, an Anti-Solvent to the Solution Obtained in Step a)

The Bortezomib solution of step a) if necessary may be combined with an anti-solvent for precipitation.

Anti-solvent that may be used in the process of the present invention include, but are not limited to, water, hydrocarbons such as toluene, xylene, cyclohexane, n-hexane, n-heptane; ethers such as diethyl ether, diisopropyl ether, tetrahydrofuran (THF), 1,4-dioxane, dimethoxyethane, methyl tertiary-butyl ether; or mixtures thereof.

The process of adding an anti-solvent and precipitating the compound may be carried out by a process as described in the above embodiment of a process for the purification of Bortezomib.

Step-c) Isolating the Solid Product from Step-a) or Step-b)

The obtained precipitate from step-a) or step-b) may be separated and dried by a process as described in the above embodiment of a process for the purification of Bortezomib.

In one embodiment, the present application provides process for the purification of Bortezomib, wherein Bortezomib is purified from a mixture of organic solvents selected from dichloromethane or ethyl acetate with toluene.

The individual solvents in the mixture may range from 1% to 99% v/v. In one embodiment, an individual solvent may range from about 5% to about 10% v/v.

An aspect of the present application provides substantially pure Bortezomib having purity of greater than about 95%, in another embodiment, greater than about 99%, in still another embodiment, greater than about 99.5% and. In still another embodiment, more than about 99.8% (by HPLC) and substantially free of its stereo isomers and/or impurities.

In one embodiment, the present application provides a process for the preparation of crystalline Form A of Bortezomib, comprising:
i) providing a solution of Bortezomib in an alcohol;
ii) adding water to precipitate the solid; and optionally
iii) separating the obtained solid.

Providing a solution of Bortezomib in alcohol includes dissolution of Bortezomib in an alcohol solvent, optionally under nitrogen atmosphere. Any crystalline or amorphous form or mixture of crystalline and amorphous forms of Bortezomib is acceptable for providing the solution.

Alcohol solvents that may be used for the dissolution include, but are not limited to methanol, ethanol, isopropyl alcohol, n-butanol, isobutanol, and t-butanol. In a particular embodiment, methanol is used for the dissolution of Bortezomib. The quantity of alcohol used for the dissolution may vary from about 2-10 ml/gm of the Bortezomib.

The solutions of Bortezomib may be provided at a temperature ranging from about 20° C. to a temperature up to the boiling point of the alcohol solvent used. In a particular embodiment, the solution of Bortezomib may be provided at a temperature about 20° C. to about 35° C. The undissolved particles may be removed suitably by filtration, centrifugation, decantation, and other techniques.

The Bortezomib solution of step i) is combined with water for precipitation. The water may be added to the solution at a temperature of about 20°-45° C., preferably, about 25°-35° C. The quantity of water used for the precipitation may vary from about 2-10 ml/g of the Bortezomib. Addition of water to the Bortezomib solution may be carried out over the period of about 5 minutes to about 1 hour or more.

The quantity of water used for precipitation depends on the concentration of Bortezomib in alcohol and the temperature of addition and may be readily determined by a person ordinary skilled in the art.

The suspension is maintained at a temperature of about 0° C. to about 35° C. In one embodiment, it is maintained at about 30°-35° C. The obtained mixture can be stirred for about 30 minutes to about 5 hours or more to affect the complete precipitation. Other methods of precipitation or complete precipitation may also be used. In one embodiment, the suspension of Bortezomib is maintained at a temperature of about 25° C. to about 35° C. for 2 to 3 hours.

The obtained precipitate may be separated by the techniques known in the art. For example it may be separated by using any techniques such as filtration by gravity or by suction, centrifugation, decantation, and the like. After separation, the solid may optionally be washed. The wet solid obtained may be dried suitably in a tray dryer, vacuum oven, air oven, fluidized bed drier, spin flash dryer, flash dryer and the like. The drying may be carried out at temperature of about 35° C. to about 70° C. and in one embodiment at about 50° C., optionally under reduced pressure. The drying may be carried out for any time period such as for about 1 to about 25 hours, or longer to get Bortezomib form A.

It was surprisingly discovered that Form A obtained by the process of the present application is a monomer rather than the trimeric anhydride.

Mass spectral analysis (positive ion, electrospray) of an acetonitrile solution of the Bortezomib form A obtained by the process of the present invention exhibited peak at m/z=383.19 in negative ion mode indicating that the product is monomeric boronic acid rather than the trimeric boroxine (anhydride form).

The positive ion mode for the same crystalline product has shown m/z=367.4 since the protonated molecular ion (M+)⁺ of Bortezomib is labile and undergoes in-source dehydration (18 Da). Further, no sodium, proton, and potassium adducts of trimeric boroxine at m/z=1121, 1099, and 1137, respectively were observed confirming the monomeric nature of the compound.

In one embodiment, the crystalline Form A of Bortezomib obtained by the process of the present application is characterized by the X-ray diffraction pattern substantially as illustrated in FIG. 1.

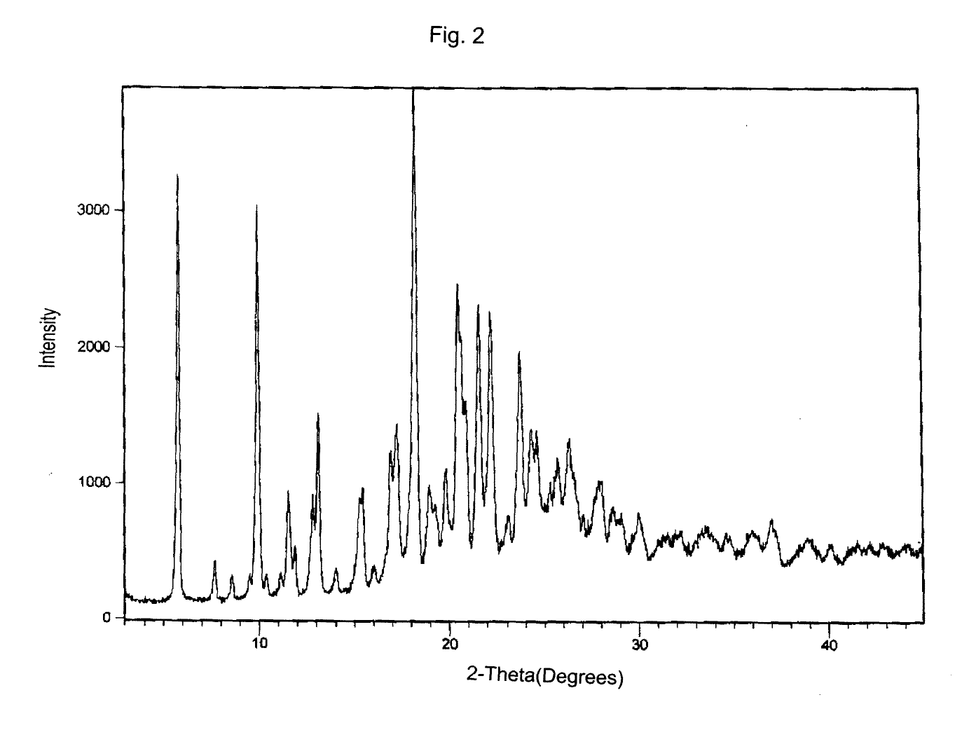

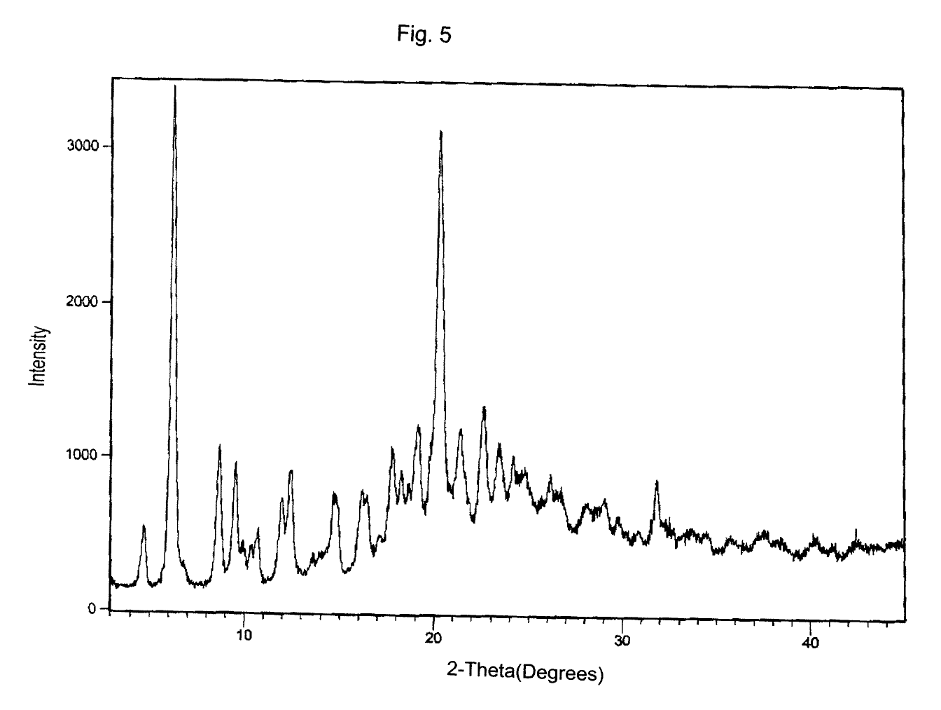

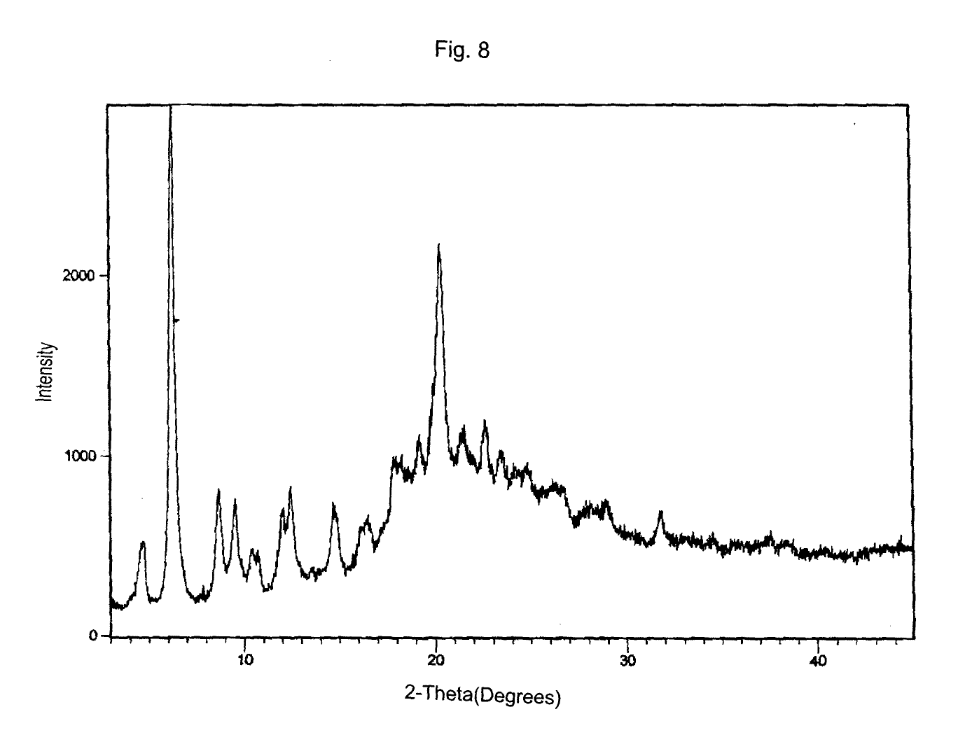

In one embodiment, the crystalline Form A of Bortezomib obtained by the process of the present application is characterized by the X-ray diffraction pattern with characteristic peaks at diffraction angles 2-theta of about 5.82, 9.47, 9.93, 12.80, 18.31, 20.50, 20.90, 21.60, 22.20, and 23.70±0.2 degrees 2-theta. This pattern, illustrated in FIG. 2, was generated using a PANalytical instrument, equipped with Bragg-Brentano theta:theta goniometer having Xcelerator*** detector. The pattern was recorded at a tube voltage of 40 kV and a tube current of 40 mA, with a step size of 0.02° and time per step of 10 sec over an angular range of 3-45° 2 theta. The sample was exposed to the CuK radiations (=1.5418 Å). This same equipment and settings were used to generate the patterns of FIGS. 5 and 8. Alternatively, Form A can be characterized by the following peaks at diffraction angles 2-theta of 5.82, 9.93, 11.53, 12.80, 13.11, 15.27, 15.47, 16.90, 17.32, 18.31, 18.96, 19.27, 19.85, 20.50, 21.60, 22.24, 23.74, 24.29, 24.65, 25.76, 26.32, 28.03, 29.96, ±0.2 degrees. A pattern reflecting these peaks is found in FIG. 1, which was generated using a Rigaku Dmax 2200 instrument, equipped with RINT2000 wide angle goniometer having Scintillation Counter detector. The pattern was recorded at a tube voltage of 50 kV and a tube current of 34 mA, with a step size of 0.02° and time per step of 3°/min over an angular range of 3-45° 2 theta. The sample was exposed to the CuK radiations (=1.5418 Å).

In another embodiment, crystalline Form A of Bortezomib obtained by the process of the present application is characterized by Differential Scanning Calorimetry (DSC) thermogram with endotherm peaks at about 75.20° C., and 179.73° C. substantially as illustrated in FIG. 2.

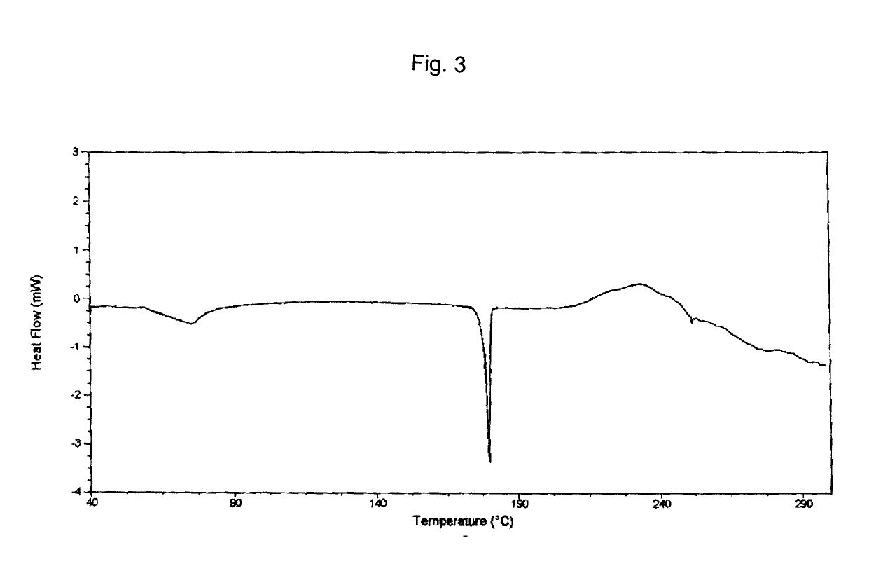

In another embodiment, crystalline Form A of Bortezomib obtained by the process of the present application is characterized by TGA curve substantially as illustrated in FIG. 3 corresponding to a weight loss of about 2.88%.

In another embodiment, crystalline Form A of Bortezomib obtained by the process of the present application is characterized by a moisture content up to about 5% by KF.

In an embodiment, the present application provides a process for the preparation of crystalline Form B of Bortezomib, comprising:
a) providing a solution of Bortezomib in a halogenated alkane solvent or a ester solvent
b) adding a aromatic hydrocarbon solvent to precipitate the solid; and optionally
c) isolating the obtained solid.

Providing a solution of Bortezomib preferably includes dissolution of the compound in either halogenated alkane solvent or an ester solvent, optionally under nitrogen atmosphere. Any crystalline form or amorphous form or mixture of crystalline and amorphous forms of Bortezomib is acceptable for providing the solution.

The quantity of solvent used for the dissolution may vary from about 2-10 ml/g of the Bortezomib.

Halogenated alkane solvents that may be used for the dissolution include, but are not limited to dichloromethane, 1,2-dichloroethane and chloroform; Ester solvents that may be used for the dissolution include, but are not limited to ethyl acetate, isopropyl acetate, tertiary butyl acetate; aromatic hydrocarbon solvents that may be used for precipitation include, but are not limited to toluene, xylenes.

Solution of Bortezomib may be provided at a temperature of about 20° C. to a temperature up to the boiling point of the solvent used. In one embodiment, the solution of Bortezomib is provided at a temperature of about 25° C. to about 35° C. The undissolved particles may be removed suitably by filtration, centrifugation, decantation, and other techniques.

Precipitation may be carried out by adding an aromatic hydrocarbon solvent to the solution of Bortezomib. The temperature at which addition may be done ranges from about 20-35° C.

The quantity of aromatic hydrocarbon used for precipitation depends on the concentration of Bortezomib in the halogenated alkane solvent or a ester solvent and the temperature of addition and may be readily determined by a person ordinary skilled in the art.

The suspension may be maintained at a temperature of about 0° C. to about 35° C. for about 30 minutes to about 5 hours or more. In one embodiment, the suspension of Bortezomib is maintained at a temperature of about 25° C. to about 35° C. for 2 to 3 hours to affect complete precipitation.

The obtained precipitate may be separated by the techniques known in the art. For example it may be separated by using any techniques such as filtration by gravity or by suction, centrifugation, decantation, and the like. After separation, the wet solid obtained may be dried suitably in a tray dryer, vacuum oven, air oven, fluidized bed drier, spin flash dryer, flash dryer and the like. The drying may be carried out at temperatures of about 35° C. to about 70° C. and preferably at about 50° C., optionally under reduced pressure. The drying may be carried out for any time periods such as for about 1 to about 25 hours, or longer, to give Bortezomib form B. Other techniques may be used as well.

In one embodiment, the crystalline Form B of Bortezomib obtained by the process of the present application is characterized by the X-ray diffraction pattern with characteristic peaks at diffraction angles 2-theta of about 4.76, 6.30, 8.69, 9.56, 10.72, 11.91, 12.45, 14.64, 16.17, 17.81, 19.21, 20.39, 21.41, 22.70, 23.40, 24.82, and 31.78±0.2 degrees 2-theta. Alternatively, Form B may be characterized by the following peaks at diffraction angles 2-theta of 4.76, 6.30, 8.69, 9.56, 10.72, 11.91, 12.45, 14.64, 16.17, 17.81, 18.27, 19.21, 20.39, 21.41, 22.70, 23.40, 24.21, 26.15, 31.78±0.2 degrees.

In another embodiment, crystalline Form B of Bortezomib obtained by the process of the present application is characterized by X-ray diffraction pattern as substantially illustrated in FIG. 5.

In another embodiment, crystalline Form B of Bortezomib obtained by the process of the present application is characterized by TGA curve substantially illustrated in FIG. 5 corresponding to a weight loss of about 0.39%.

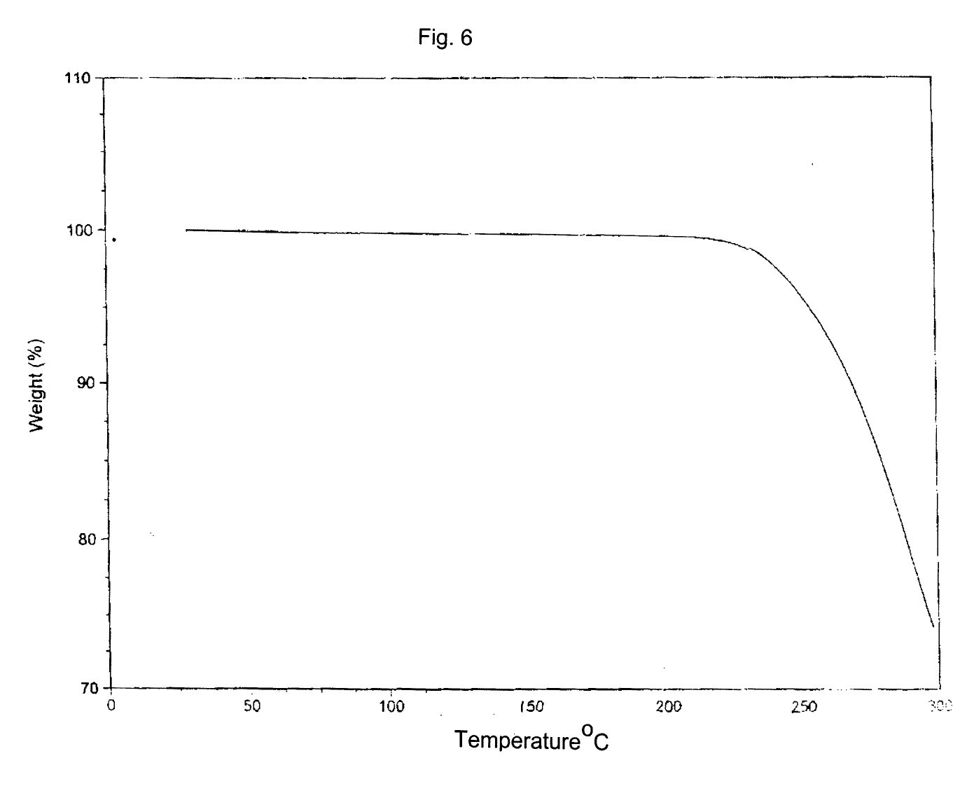

In another embodiment, crystalline Form B of Bortezomib obtained by the process of the present application is characterized by an infrared absorption spectrum in a potassium bromide (KBr) pellet as substantially illustrated by the spectrum of FIG. 6.

In another embodiment, crystalline Form B of Bortezomib obtained by the process of the present application is characterized by moisture content up to about 3% by KF.

The residual solvents in Bortezomib, including forms A and B, obtained by the processes of the present application is within the limits given by the International Conference on Harmonization of Technical Requirements for Registration of Pharmaceuticals for Human Use (“ICH”) guidelines.

Bortezomib obtained by the processes of the present application may optionally be milled to get the required particle size. Milling or Micronization may be performed prior to drying, or after the completion of drying of the product. The milling operation reduces the size of particles and increases the surface area of particles by colliding particles with each other at high velocities.

In another embodiment, the present application provides a pharmaceutical composition containing a pharmaceutically effective amount of crystalline forms A and/or B of Bortezomib obtained by the processes of the present invention and at least one pharmaceutically acceptable excipient.

Bortezomib crystalline forms obtained as per the processes of the present application are not only stable but also well suited for use in preparing pharmaceutical formulations. The pharmaceutical formulations according to the present application include but are not limited to solid oral dosage forms such as tablets, capsules, powders and so on; liquid oral dosage forms such as solutions, dispersions, suspensions, emulsions and so on; parenteral dosage forms (including intramuscular, subcutaneous, intravenous) such as injectable dosages by solution or suspension or dispersions or sterile powders for reconstitution; transdermally delivery systems; targeted delivery systems etc.

Further, the inventors of the present application have found that Bortezomib stored in a storage system comprising a sealed polymeric bag, (e.g., a transparent or opaque polyethylene bag without desiccants) or a combination of such bags under normal temperature conditions is found to be unstable. Stability Study of Bortezomib when stored under general storage conditions is summarized in Table 1.

TABLE 1
Bortezomib when stored at 25° C. and 60% RH (General
storage conditions and without using the conditions
described herein as an aspect of the invention

| | Initial | Purity on | Purity on | Purity on |
|---|---|---|---|---|
| Compound | purity | 7^{th} day | 15^{th} day | 30^{th} day |
| Bortezomib* | 99.49% | 99.34% | 98.92% | 97.22 |
| purity by HPLC | | | | |
| Impurity-a | 0.06% | 0.06% | 0.08% | 0.18% |
| Impurity-b | 0.36% | 0.33% | 0.35% | 0.39% |

*from ethylacetate
wherein, Impurity-a is

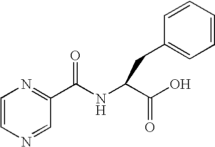

and
Impurity-b is the combination of RR and SS diastereomers of Bortezomib.
(S,S)-isomer:

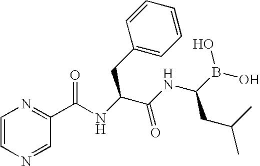

(R,R)-isomer:

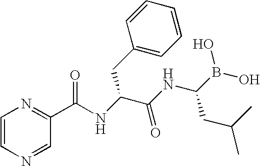

The foregoing results show that Bortezomib stored at 25° C. and 60% RH in a storage system as described above shows substantial degradation of product, which resulted in decrease in the purity of Bortezomib.

It has now been found that storing Bortezomib as per the present application method of storage in a controlled environment stabilizes the Bortezomib and maintains the purity of Bortezomib over long storage periods. Without wishing to be bound by any particular theory, it is believed that the apparent instability problems associated with Bortezomib may be overcome by storing Bortezomib in an environment having reduced humidity levels, and/or low atmospheric oxygen levels and/or low light levels.

In a further aspect, the present application provides a storage system for stabilizing Bortezomib. The storage system of the present application preferably comprises at least one sealed polymeric bag, (e.g. a transparent or opaque polyethylene bag-having thickness of about 0.10 mm to about 0.50 mm) or a combination of such bags, which, if desired, may be sealed inside of a laminated aluminum bag. An oxygen absorbent and a moisture absorbent (or desiccant), may be included between one or more of such bags. Finally, the packed samples are stored in HDPE containers.

In one of the preferred aspect, the present application provides a storage system for stabilizing Bortezomib under inert atmosphere, wherein the packaging system comprising of:
a. At least one external sealed polymeric bag;
b. A separate polymeric bag containing Bortezomib or optionally a combination of such bags, which, if desired, may be sealed inside of a laminated aluminum bag;
c. An oxygen absorbent and optionally a moisture absorbent (or desiccant) interposed between polybag a. and polybag b.

The storage system of the present application preferably includes a container with Bortezomib contained therein, wherein the container is preferably capable of providing an internal environment having lower humidity, oxygen and light levels, or a combination thereof, relative to the external environment. The storage system of the present application is preferably capable of maintaining the Bortezomib purity for at least about 3 months under storage condition (at a temperature of 2-8° C. and also at 25 to 35° C., 60% RH). The storage system of the present application is more preferably capable of maintaining the Bortezomib purity for at least about 3 months and most preferably for about 6 months with a maximum degradation of less than about 0.2% from the initial purity or free from any degradation. Stability experiments conducted for Bortezomib obtained by the process of the present application in a controlled environment are summarized in Table-2.

In a particularly preferred embodiment, the storage system of the present application is capable of maintaining Bortezomib, e.g. Bortezomib produced according to Example 5, for at least about one month, at least about two months, or even at least about 6 months at 2-8° C. or more (Please refer Table-2).

The container, which may be used in the storage system of the present application, preferably includes at least one external sealed polymeric bag. Suitable polymeric bags may include one or more commercial bags suitable for storing purposes, e.g., polyethylene bags (e.g., low density polyethylene bags and high density polyethylene bags), polypropylene bags, polyester bags, nylon bags, polyvinyl chloride (PVC) bags, and the like. The polymeric bags utilized in the storage system may have thickness of about 0.10 mm to about 0.50 mm.

In a particularly preferred embodiment, the storage system includes a sealed laminated aluminum bag, high density polyethylene bag contained within the sealed aluminum bag, a sealed transparent low density polyethylene bag contained within the sealed opaque polyethylene bag, Bortezomib contained within the transparent polyethylene bag, and an oxygen absorbent, and optionally a desiccant or both interposed between the transparent and opaque polyethylene bags.

Suitable oxygen absorbents include but are not limited to organic types, based on ascorbic acid and inorganic types based on iron powder containing materials. Preferably AgelessZ200 or AgelessZ100 or the like may be utilized may as oxygen absorbent for maintaining the stability of Bortezomib.

Suitable desiccants include but not limited to aluminum oxide, calcium chloride, Drierite (CaSO₄), molecular sieves (e.g., activated molecular sieves), silica gel, and the like, and combinations thereof. Preferably silica gel may be used as desiccant for maintaining the stability of Bortezomib.

The drug packaging of the present application may prevent the degradation of Bortezomib over long storage periods. Preferably, the storage system is capable of reducing or eliminating drug instability due to contact with oxygen and/or water. This system results in eliminating significant degradation to the Bortezomib, more preferably a maximum degradation of up to about 0.2% from the initial purity or free from any degradation, when stored for minimum for about three months.

Exemplary packaging for the storage system of the present application also may include a packaging type, wherein Bortezomib is packed and sealed in a transparent polyethylene bag, which is packed in an opaque (e.g., black) polyethylene bag, which is then sealed and, in turn, packed and sealed in a laminated aluminum bag, which is then sealed. An oxygen absorbent and a desiccant may be interposed (e.g., dispersed) between the two polymeric bags/layers.

In accordance with the present application, packaging as described herein, may be packed under an inert atmosphere (e.g., under a nitrogen atmosphere) or in ambient air.

Certain specific aspects and embodiments of the present application will be explained in more detail with reference to the following examples, which are provided by way of illustration only and should not be construed as limiting the scope of the invention in any manner.

### EXAMPLES

### Example-1

### Process for Preparing N-[(1S)-2-[[(1R)-1-[(3aS,4S,6S,7aR)-hexahydro-3a,5,5-trimethyl-4,6-methano-1,3,2-benzodioxaborol-2-yl]-3-methyl butylamino]-2-oxo-1-(phenylmethyl)ethyl] Pyrazinecarboxamide (Formula IX)

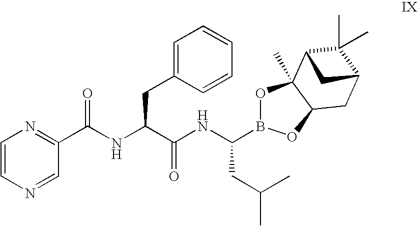

The process for preparing compound of formula IX comprises of the steps from Step a) to step h), which are individually demonstrated below:

### Step-a) Preparation of 2-(2-Methylpropyl)-(3aS,4S,6S,7aR)-hexahydro-3a,5,5-trimethyl-4,6-methano-1,3,2-benzodioxaborole (Formula II)

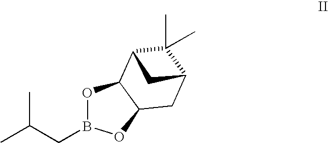

To a stirred solution of isobutyl boronic acid (50.0 g) in n-heptane (250 ml) at 25-30° C., was added (+)-Pinanediol (83.3 g) and stirred for 1 hour at 25-30° C. To the reaction mass was added brine solution and the mixture was stirred. The layers were allowed to separate and the organic layer was concentrated under reduced pressure till no more solvent distills off to give the title compound (Formula II).

### Step-b) Preparation of 2-((1S)-1-Chloro-3-methylbutyl)-(3aS,4S,6S,7aR)-hexahydro-3a,5,5-trimethyl-4,6-methano-1,3,2-benzodioxaborol (Formula III)

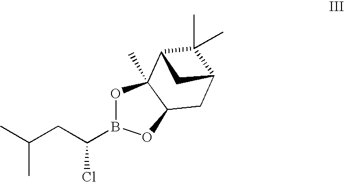 [0235] I. preparing a mixture of zinc chloride with tetrahydrofuran
II. preparing LDA mixture
III. preparing a solution of compound of formula-II

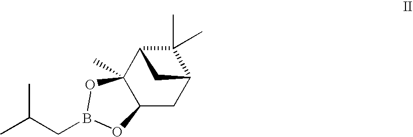

in a solvent mixture comprising dichloromethane and water miscible ether solvent
IV. adding solution of step II into the solution of step III followed by maintaining the solution at a temperature of about −40 to −70° C.
V. adding the mixture of step I into the product of step 1V followed by maintaining the reaction mass at a temperature of about −40 to −70° C.
VI. raising the reaction temperature up to about 10° C. to ambient temperature
VII. adding the aqueous acid solution
VIII. separating the organic layer containing the compound of formula-III, and isolating the product.

I. Preparing a Mixture of Zinc Chloride with Tetrahydrofuran

Charged ZnCl₂ (115 g) to tetrahydrofuran (805 ml) into a 1^{st} Round bottom flask (R.B. flask) under nitrogen atmosphere at 25 to 35° C. and the temperature of the resulting mixture was raised to 35 to 40° C., maintained for 3-4 hours to give ZnCl₂ solution.
II. Preparing LDA Mixture

Charged diisopropyl amine (86 ml) to tetrahydrofuran (345 ml) into a 2^{nd} R.B. flask under nitrogen atmosphere and resultant mixture was cooled to −7 to −15° C., charged n-hexyl lithium to the above mixture and maintained for 30-40 minutes to give LDA mixture.

III. Preparing a Solution of Compound of Formula-II

Compound of Formula II (115.0 g) was charged to a dichloromethane (161 ml) and tetrahydrofuran (690 ml) into a 3^{rd} R.B. flask under nitrogen atmosphere at 25 to 35° C. and the mixture was cooled to −55 to −60° C.

IV. Adding Solution of Step II into the Solution of Step III

Charged LDA mixture from the 2^{nd} R.B. flask to the reaction mixture at −55 to −60° C. and maintained for 30 minutes. The temperature was raised to −50° C.

V. Adding the Mixture of Step I into the Product of Step IV

Charged ZnCl₂ solution from the 1^{st} R.B. flask at −45 to −50° C. and maintained for 1 hour.

VI. Raising the Reaction Temperature Up to about 10° C.

The reaction mixture was warmed to 10° C.

VII. Adding the Aqueous Acid Solution

Charged 10% H₂SO₄, stirred for 10-15 minutes and the organic layer was separated.

VIII. Separating the Organic Layer Containing the Compound of Formula-III, and Isolating the Product.

The organic layer separated under step VII was subjected to next step, however, the aqueous layer was discarded.

Washed the organic layer with brine solution under stirring, till the aqueous layer reached to a pH around 6-7.

The organic layer was concentrated to isolate the compound of formula-III, under reduced pressure.

### Step-c) Preparation of N,N-Bis(trimethylsilyl)-(1R)-1-[(3aS,4S,6S,7aR)-hexahydro-3a,5,5-trimethyl-4,6-methano-1,3,2-benzodioxaborol-2-yl]-3-methylbutylamine (Formula IV)

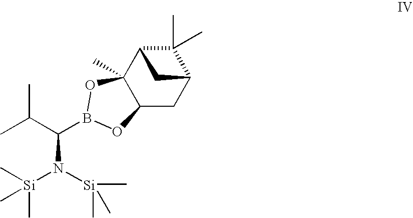

Hexamethyldisilazane (101.3 ml) was charged to tetrahydrofuran (414 ml) under nitrogen atmosphere and the mixture was cooled to −20 to −30° C. Charged n-hexyllithium slowly to the above mixture under stirring by maintaining the temperature at −20 to −30° C. The reaction mixture was stirred for 1-2 hours at −20 to −25° C., charged compound of Formula III (138 g) to the above freshly prepared lithium HMDS in THF by maintaining the temperature at −15 to −20° C. The reaction mixture was warmed to a temperature of 25-30° C. and maintained for 2-3 hours. Filtered the reaction mixture through silica bed and washed the bed with diisopropyl ether. The filtrate was concentrated under reduced pressure to a residue to give the title compound (Formula IV).

### Step-d) Preparation of 4,6-Methano-1,3,2-benzodioxaborole-2-methanamine, hexahydro-3a,5,5-trimethyl-α-(2-methylpropyl)-,(αR,3aS,4S,6S,7aR)-,trifluoro acetate (Formula V)

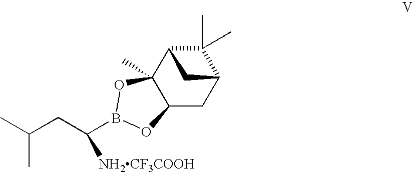

Charged trifluoroacetic acid (129 ml) to diisopropyl ether (1980 ml) under nitrogen atmosphere at 25-30° C. and the reaction mass cooled to −10° C. Charged compound of Formula IV (198 g) to the reaction mass slowly at −10° C. and maintained at the same temperature for 8 hours. The reaction mass was filtered, washed with diisopropyl ether (198 ml) and the obtained solid was slurry washed with water (1500 ml) at 25-30° C. The slurry was filtered washed with water and the solid obtained was dried at 40-50° C. under reduced pressure for 8 hours to give 74.0 g of the title compound (Formula V).

### Purity (by GC)=99.54%

### Step-e) Preparation of L-Phenylalanine methyl ester hydrochloride (Formula VI)

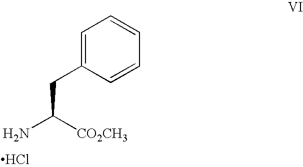

To a stirred mixture of L-phenyl Alanine (25 g) in methanol (125 ml) at 25-30° C., was charged thionyl chloride (13.2 ml) under stirring and the mixture was maintained at 55-60° C. for 2-3 hours. The reaction mass was cooled to 25-30° C. and concentrated under reduced pressure up to 2 volumes with respect to the staring material. Charged isopropyl alcohol (125 ml) to the reaction mass and concentrated up to 2 volumes with respect to the starting material. Cooled the reaction mass to 0-5° C. and maintained under stirring for 1-2 hours. Filtered the reaction mass, washed with isopropyl alcohol, suck dried for 30 minutes and the solid obtained was dried at 45-50° C. for 3-4 hours to give 28.8 g of the title compound (Formula VI). Chiral purity by HPLC: 100%.

### Step-f) Preparation of L-Phenylalanine, N-(pyrazinylcarbonyl)-methyl ester (Formula VII)

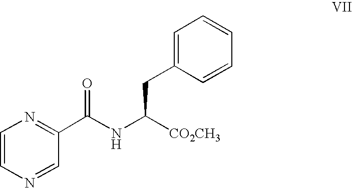

To a stirred mixture of Pyrazine-2-carboxylic acid (3.45 g) in DMF (50 ml) at 25-30° C., was charged N-hydroxy succinimide (3.2 g) under stirring and was cooled to 0-5° C. Charged N,N′-dicyclohexylcarbodiimide (DCC) (5.75 g) to the reaction mass at 0-5° C. and stirred for 15-20 minutes. Charged compound of Formula VI (5.0 g) to the reaction mass at 0-5° C. and stirred for 15-20 minutes. Further, charged NMM (3.8 ml) to the reaction mass at 0-5° C. and stirred for 15-20 minutes. The reaction mixture was warmed to 25-30° C. and maintained under stirring for 2-3 hours. The reaction mass was filtered and the solid was separated. The filtrate obtained was diluted with ethylacetate (100 ml) and washed with demineralized water. The organic layer was washed with 1N HCl, followed by washing with sodium bicarbonate solution. Concentrated the organic layer up to 2 volumes with respect to Formula VI under reduced pressure and cooled to 25-30° C. Charged n-heptane (20 ml) to precipitate the compound, cooled the reaction mass to 0-5° C., maintained for 1-2 hours and filtered under vacuum. The solid obtained was dried at 40-45° C. for 3-4 hours to give 5.7 g of the title compound (Formula VII). Purity by HPLC: 99.73%, chiral purity by HPLC: 99.97%.

### Step-g) Preparation of N-(pyrazinylcarbonyl)-L-Phenylalanine (Formula VIII)

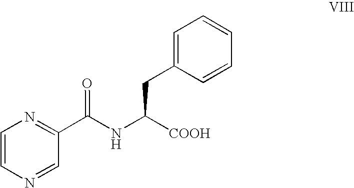

To a stirred mixture of Formula VII (100 g) in acetone (500 ml) at 25-30° C., was charged NaOH solution (obtained by dissolving 15.4 g of NaOH in 500 ml of water) and maintained at the same temperature for 30-50 minutes. Adjusted the pH of the reaction mass to 2 by using 1N HCl and cooled the reaction mass to 0-5° C. Maintained the reaction mass at 0-5° C. under stirring for 1-2 hours, filtered under vacuum and dried the material obtained at 45-50° C. for 4-5 hours to give 84.4 g of the title compound (Formula VIII).

Purity by HPLC: 99.94% by weight.
Chiral purity by HPLC: 100%
Alternately, N-(pyrazinylcarbonyl)-L-Phenylalanine (Formula VIII) may also be prepared by

(a) Using ethylchloroformate according to the process as described below:

A mixture of acetone (40 ml), pyrazine carboxylic acid (5 g) and triethylamine (6.77 ml) was cooled to about −5° C. to about 0° C. and ethylchloroformate (4.76 ml) was charged. The reaction mass was stirred for about 30 minutes. The reaction suspension was allowed to reach the temperature of about 25° C. to about 30° C. and maintained for about 3 hours. The reaction suspension was cooled to about 0° C. to about 5° C. In the second flask the aqueous sodium hydroxide (1.68 g in 70 ml water) solution was cooled to about 0° C. to about 5° C. and to that acetone (30 ml) and L-phenyl alanine (6.6 g) were added and the mixture was stirred for about 1 hour at that temperature. The reaction mass of the second flask was added to the reaction mass of the first flask at a temperature of about 0° C. to about 5° C. and then stirred for about 2 hours followed by raising the temperature to about 25° C. to about 30° C. The reaction mass was further stirred for about 16 hours at a temperature of about 25° C. to about 30° C. Ethyl acetate (150 ml) was charged to the reaction solution and stirred for about 30 minutes. The layers were separated and 1N hydrochloric acid (35 ml) was added to the separated aqueous layer. The reaction solution was cooled to about 0° C. to about 5° C. and stirred for about 2 hours. The obtained suspension was filtered and the solid was washed with water (10 ml). The solid was then dried at a temperature of about 50° C. for about 4 hours to afford 2.6 g of title compound.

Purity by HPLC: 99.2% by weight.
Chiral purity by HPLC: 100%

(b) or by using combination of EDC.HCl, HOBt, according to the process as described below:

A mixture of pyrazine carboxylic acid (168.7 g), dimethylformamide (1.4 lit), hydroxybenzotriazole (HOBt:220 g), and N-methyl morpholine (221 ml) was cooled to a temperature of about 0° C. to about 5° C. EDC.hydrochloride (1-Ethyl-3-(3-dimethylaminopropyl)carbodiimide-HCl; 278 g) was added to the reaction solution at a temperature of about 0° C. and stirred for about 30 minutes. L-phenylalanine methyl ester hydrochloride (240 g) obtained from above was dissolved in DMF (1 lit) and then added to the reaction mixture. N-methyl morpholine (110 ml) was added to the reaction mixture and the reaction mixture was maintained at a temperature of about 0° C. to about 5° C. for about 1 hour. The reaction mixture was allowed to warm to the temperature to about 25° C. to about 35° C. and diluted with water (3.6 lit). The reaction mass was extracted with ethyl acetate (3×2.4 lit). The separated ethyl acetate layer was washed with 1N hydrochloric acid (1.2 lit) and two layers were then separated. The organic layer was washed with saturated sodium bicarbonate solution (4.8 lit) and brine solution (2.4 lit). The organic layer was concentrated completely at a temperature of about 45° C. to afford 260 g of pyrazine-2-carbonylphenylalanine methyl ester.

Pyrazine-2-carbonylphenylalanine methyl ester (5 g) was dissolved in acetone (25 ml) and stirred for about 5 minutes. Sodium hydroxide solution (701 mg of sodium hydroxide in 25 ml of water) was added to the reaction solution and stirred for about 3 hours at a temperature of about 25° C., and the pH was then adjusted with 1N hydrochloric acid (11 ml) to a pH of about 2. The reaction mixture was cooled to a temperature of about 0° C. to about 5° C. and stirred for about 1 hour. The suspension was filtered and suck dried to afford 4.0 g of pyrazine-2-carbonylphenylalanine.

Chiral purity by chiral HPLC: 100%
Chemical purity by HPLC: 99.88%.

### Step-h) Preparation of Formula IX

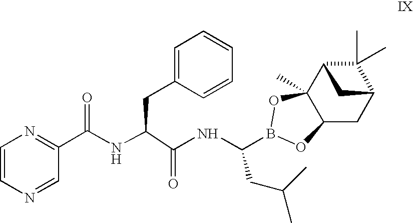

To a stirred mixture of compound of Formula VIII (28.6 g) in dichloromethane (400 ml) at 25-30° C. under nitrogen atmosphere, were charged N-hydroxysuccinimide (13.3 g) and DCC (23.9 g) and stirred for 10-20 minutes. Charged compound of Formula-V (40 g) to the reaction mass was and stirred for 15-20 minutes.

Charged diisopropylethylamine (DIPEA) (27 ml) and maintained the reaction mass at 25-30° C. for 2-3 hours. The reaction mass was filtered and the solid was washed with dichloromethane (80 ml). The filtrate obtained was washed with 1N HCl, followed by washing with sodium bicarbonate solution. Concentrated the organic layer up to 2 volumes with respect to Formula-V. Charged methanol (200 ml) and concentrated up to 2 volumes with respect to Formula-V. The concentrated mass obtained is the title compound (Formula IX).

Alternately, compound of Formula IX may also be prepared by using EDC.HCl, and Hydroxybenzotriazole by a process as described below:

N-(2-pyrazinecarbonyl)-L-phenylalanine (500 mg) was suspended in dichloromethane (10 ml) and cooled to about −5° C. to about 0° C. Hydroxybenzotriazole (HOBt:310 mg) was charged in to the reaction mass followed by the addition of 1-ethyl-3-(3-dimethylaminopropyl) carbodiimide hydrochloride (EDC.HCl, 385 mg) and stirred for 15 minutes. (1R)-(S)-pinanediol 1-ammonium trifluoroacetate-3-methylbutane-1-boronate (695 mg) was added to the reaction mixture and stirred for about 10 minutes at a temperature of about 5° C. Diisopropyl ethyl amine (0.6 ml) was charged to the reaction mixture and stirred for about 30 minutes at a temperature of about 5° C. The reaction mixture was allowed to warm to a temperature of about 25° C. to about 30° C. and stirred for about 1 hour followed by the addition of 1N hydrochloric acid (30 ml). The layers were separated and the organic layer was washed with 1N hydrochloric acid (15 ml) and saturated sodium bicarbonate solution (2×30 ml). The organic layer was concentrated completely to afford title compound.

Purity by HPLC: 84.98% Note that it is believed that 9.01% measured by HPLC is Bortezomib that is formed prior to the final Bortezomib step. Thus, overall purity should be 84.98%+9.0% or 93.99% as measured by HPLC.

### Example-2

### Process for Preparing Bortezomib (Formula I)

To a stirred mixture of compound of Formula IX (13.6 g) in methanol (272 ml) at 25-30° C., was charged n-heptane (272 ml), and isobutylboronic acid (3.2 g). Charged 2N HCl (272 ml) to the reaction mass under vigorous stirring and maintained the reaction mass at 25-30° C. for 1-2 hours. After the completion of the reaction, separated the n-heptane layer and discarded. Charged n-heptane (272 ml*2) to the aqueous layer and stirred vigorously for 10-15 minutes. Separated the n-heptane layer and the aqueous layer obtained was concentrated under vacuum at 35 to 48° C. The aqueous layer was extracted with dichloromethane (272 ml) under vigorous stirring. The extraction process is repeated (272 ml*2) and the obtained dichloromethane layers were pooled and washed with saturated sodium bicarbonate solution, followed brine solution. The organic layer is separated, concentrated under vacuum to give 6 ml of the reaction mass and allowed to cool to 25-30° C.

### Purity: 95.13% by HPLC.

Charged Toluene (102 ml) to the above reaction mass and stirred at 25-30° C. for 2-3 hours. Filtered the solid obtained under vacuum washed with 5% dichloromethane in toluene and dried at 45-50° C. under vacuum for 5 hours to give crude Bortezomib.

### Yield: 7.0 g (70%)

### Purity by HPLC: 99.22%

### Impurity-B by HPLC: 0.43%

### Polymorphic Form Form-B

### XRD Pattern: As Illustrated in FIG. 5

### Example-3

### Process for Purification of Bortezomib Using Methanol and Water

Bortezomib (5.0 g, purity 99.22%) and methanol (15 ml) were taken into a round bottom flask and stirred at 25 to 35° C. Demineralized water (15 ml) was added to the obtained solution and stirred for 2 hours at a temperature of about 27° C. The reaction suspension was filtered and washed the solid with aqueous methanol (30 ml; water:methanol 1:1). The obtained solid was dried at a temperature of about 50° C. for about 5 hours to afford 3.4 g of title compound.

### Purity by HPLC: 99.57%

### Impurity-B by HPLC: 0.30%

Further purification of the product obtained by reproducing the same process resulted in a Bortezomib having a purity of 99.6% by HPLC.

### Impurity-B by HPLC: 0.23%

### Chiral Purity by HPLC: 99.83%

### Example-4

### Process for Preparing Bortezomib Followed by Purification

To a stirred mixture of compound of formula IX (68.3 g) in methanol (1.22 L) at 25-30° C., was charged n-heptane (1.36 L), and isobutylboronic acid (16.13 g). Charged 1N HCl (13.6 L) to the reaction mass under stirring and maintained the reaction mass at 25-30° C. for 1-2 hours. After the completion of the reaction, separated the n-heptane layer and discarded. Charged n-heptane (1.36 L*2) to the aqueous layer and stirred vigorously for 10-15 minutes. Separated the n-heptane layer and the aqueous layer obtained was concentrated under vacuum. The aqueous layer was extracted with dichloromethane (13.6 L) under vigorous stirring. The extraction process is repeated (13.6 L*2) and the obtained dichloromethane layers were pooled and washed with saturated sodium bicarbonate solution, followed by brine solution. The organic layer was separated, concentrated under vacuum to give crude Bortezomib (47.0 g)

### Purity by HPLC: 95.62%

### Impurity-B by HPLC: 0.59%

sPurification 1: Bortezomib (25 g, Purity: 95.62%) and 5% ethylacetate in Toluene (250 ml) were taken into a round bottom flask and stirred at 25 to 35° C. for 2-3 hours. Filtered the solid obtained under vacuum washed with 5% ethylacetate in toluene and dried at 50° C. under vacuum for 5 hours to give Bortezomib.

### Yield: 18.0 g (72%)

### Purity by HPLC: 99.68%

### Impurity-B by HPLC: 0.27%

Purification 2: Bortezomib (18.0 g, purity 99.68%) and methanol (54 ml) were taken into a round bottom flask and stirred. Filtered the reaction mass through scinted funnel and washed the bed with 18 ml methanol. Demineralized water (72 ml) was added to the obtained filtrate and stirred for 2 hours at a temperature of about 27° C. The reaction suspension was filtered and washed the solid with aqueous methanol (108 ml; Water:methanol 1:1). The obtained solid was dried at a temperature of about 50° C. for about 5 hours to afford 14 g of title compound.

### Yield: 14.0 g (77%)

### Purity by HPLC: 99.83%

### Impurity B: 0.15% (by HPLC)

### Chiral Purity by HPLC: 99.85%

### Example-5

### Process for Preparing Bortezomib Followed by Purification

To a stirred mixture of compound of formula IX (10.25 g) in methanol (174.5 ml) at 25-30° C., was charged n-heptane (205 ml), and isobutylboronic acid (2.42 g). Charged 0.5N HCl (205 ml) to the reaction mass under stirring and maintained the reaction mass at 25-30° C. for 1-2 hours. After the completion of the reaction, separated the n-heptane layer and discarded. Charged n-heptane (205 ml*2) to the aqueous layer and stirred vigorously for 10-15 minutes. Separated the n-heptane layer and the aqueous layer obtained was concentrated under vacuum. The aqueous layer was extracted dichloromethane (205 ml) under vigorous stirring. The extraction process is repeated (205 ml) and the obtained dichloromethane layers were pooled and washed with saturated sodium bicarbonate solution, followed brine solution. The organic layer is separated, concentrated under vacuum to give crude Bortezomib (5.8 g).

### Purity by HPLC: 95.81%

### Impurity-B by HPLC: 0.34%

sPurification 1: Bortezomib (5 g, Purity: 95.81%) and 5% dichloromethane in Toluene (40 ml) were taken into a round bottom flask and stirred at 25 to 35° C. for 2-3 hours. Filtered the solid obtained under vacuum, washed with 5% dichloromethane in toluene and dried at 50° C. under vacuum for 5 hours to give Bortezomib.

### Yield: 4.2 g (84%)

### Purity by HPLC: 99.12%

### Impurity-B by HPLC: 0.31%

Purification 2: Bortezomib (4.2 g, purity 99.12%) and methanol (12.6 ml) were taken into a round bottom flask and stirred. Demineralized water (12.6 ml) was added to the reaction mass and stirred for 2 hours at a temperature of about 27° C. The reaction suspension was filtered and washed the solid with aqueous methanol (25.2 ml; water:methanol 1:1). The obtained solid was dried at a temperature of about 50° C. for about 5 hours to afford 2.95 g of title compound.

### Yield: 2.95 g (70%)

### Purity by HPLC: 99.70%

### Impurity-B by HPLC: 0.2%

### Chiral Purity: 99.83% (by HPLC)

### Example-6

### Process for Purification of Bortezomib Using Ethylacetate and Toluene

Bortezomib (5.0 g, Purity: 96.0%) and 5% ethylacetate in toluene (40 ml) were taken into a round bottom flask. The reaction mixture was stirred for 3 hours at a temperature of about 28° C. The reaction mixture was filtered and washed the solid with 5% ethylacetate in toluene (50 ml). The obtained solid was dried at 50° C. for 5 hours to afford 3.5 g of title compound.

### Purity by HPLC: 99.28%

Isomeric impurity by HPLC: 0.55%

### XRD Pattern: As Illustrated in FIG. 8

### Example-7

### Process for Purification of Bortezomib Using Isopropyl Alcohol and Diisopropyl Ether

Bortezomib (1.0 g, Purity: 93.46%) and isopropyl alcohol (6.0 ml) were taken into a round bottom flask and stirred at about 27° C. for dissolution. Diisopropyl ether (20 ml) was added to the obtained solution and stirred for 3 hours at a temperature of about 26° C. The reaction mixture was filtered and washed the solid with diisopropyl ether (5 ml). The obtained solid was suck dried for about 15 minutes to afford 400 mg of title compound.

### Purity by HPLC: 99.49%

Isomeric impurity by HPLC: 0.18%

### Example-8

### Process for Purification of Bortezomib

Bortezomib (0.5 g, Isomeric impurity 1.89%; Purity: 97.47%) and methanol (1.5 ml) were taken into a round bottom flask. Water (1.5 ml) was added to the obtained solution and stirred for about 2 hours at a temperature of about 25° C. The reaction suspension was filtered and the solid was washed with aqueous methanol (12 ml; water+methanol 1:1). Finally, the obtained solid was suck dried at a temperature of about 25° C. for about 20 minutes to afford 400 mg of title compound.

Purity by HPLC: 99.35% by weight
Isomeric purity by HPLC: 0.65% by weight.

### Optical Rotation:

SOR (specific optical rotation): −45.00° at 25° C. on as is basis in the medium of 5N
HCl (concentration: 1%).
SOR: −43.82° at a temperature of 25° C. on as is basis in the medium of methanol (concentration: 1%)

### XRD Pattern: As Illustrated in FIG. 1.

### Example-9

### Process for the Preparation of Form-A of Bortezomib

Bortezomib (3.0 g, purity 99.57%) and methanol (9 ml) were taken into a round bottom flask and stirred at 25 to 35° C. Demineralized water (9 ml) was added to the obtained solution and stirred for 2 hours at a temperature of about 27° C. The reaction suspension was filtered and washed the solid with aqueous methanol (18 ml; water:methanol 1:1). The obtained solid was dried at a temperature of about 50° C. for about 5 hours to afford 2.0 g of title compound.

### XRD Pattern: As Illustrated in FIG. 2

### Example-10

### Stability Study of Bortezomib in an Exemplary Storage System of the Present Invention

Two different samples of Bortezomib were stored at a storage condition of 2-8° C. and 25° C., 60% relative humidity (RH) in a storage system (packed in one Polybags having a thickness about 0.10 mm followed by repacking in another polybag having thickness of about 0.10 mm and containing external desiccant). A small quantity was taken from each sample after 15 days, 1 month (30^{th} day), 2 months (60^{th} day) and 3 months (90^{th} day) and the purity was checked by HPLC and compared to the original HPLC chromatogram of each sample (before starting the storage period). The chemical purity of each sample was obtained as % area by the HPLC chromatogram and compared to the initial purity value (before starting the storage period). The results are summarized in Table 2.

TABLE 2
Stability of Bortezomib when stored at 2-8° C. and 25° C.
and 60% RH in packaging as described in the present invention

| Storage | Initial | Purity on | Purity on | Purity on | Purity on |
|---|---|---|---|---|---|
| Conditions | purity | 15^{th} day | 30^{th} day | 60^{th} day | 90^{th} day |
| Stored at | 99.60% | 99.59% | 99.59% | 99.65% | 99.66% |
| 2-8° C. * | | | | | |
| Stored at | 99.58% | 99.56% | 99.60% | 99.63% | 99.54% |
| 25° C. and | | | | | |
| 60% RH ** | | | | | |

* from methanol/water (Form A)
** from ethylacetate

The foregoing results show that Bortezomib can be stabilized while retaining its purity content over prolonged storage periods using packaging conditions as described by the present invention.

### Example-11

### Stability Study of Aqueous Solution of Bortezomib in Basic Medium

The aqueous solution of Bortezomib is obtained from the reaction process as described in prior art and adjusted to a basic pH ˜10.5 (using 2N NaOH) and maintained at 25 to 35° C. A small quantity was taken from solution after 1 hours, 2 hours and 3 hours and the purity was checked by HPLC and compared to the original HPLC chromatogram of initial sample (immediately after adjusting the pH). The purity of each sample was obtained as % area by the HPLC chromatogram and compared to the initial purity value. The results are summarized in Table 3.

TABLE 3
Degradation of the Bortezomib in basic medium with time

| | | After | After | After |
|---|---|---|---|---|
| | Initial | 1 hour | 2 hour | 3 hours |
| Chemical Purity | 98.61% | 98.24% | 98.01% | 97.77% |
| by HPLC | | | | |
| Impurity-a | 0.7899% | 0.8924% | 0.9587% | 1.0229% |
| Impurity-b | 0.2991% | 0.4005% | 0.4746% | 0.5418% |

wherein Impurity a is

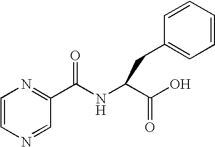

and
Impurity b is the combination of RR and SS diastereomers of Bortezomib.

From the above data it is apparent that the purity of the compound decreased continually and the content of impurities increased gradually with time, when the aqueous solution of Bortezomib was maintained under stirring in a basic medium.

### Example-12

### Process for Preparing Bortezomib Followed by Purification

To a stirred mixture of compound of formula IX (27.3 g) in methanol (491.4 ml) at 25-30° C., was charged n-heptane (546 ml), and isobutylboronic acid (6.4 g). Charged 1.0 N HCl (546 ml) to the reaction mass under stirring and maintained the reaction mass at 25-30° C. for 1-2 hours. After the completion of the reaction, separated the n-heptane layer and discarded. Charged n-heptane (546 ml*2) to the aqueous layer and stirred vigorously for 10-15 minutes. Separated the n-heptane layer and the aqueous layer obtained was concentrated under vacuum to remove methanol. The aqueous layer was extracted with dichloromethane (546 ml) under vigorous stirring. The extraction process is repeated with dichloromethane (546 ml×2) and the obtained dichloromethane layers were pooled and washed with saturated sodium bicarbonate solution, followed brine solution. The organic layer is separated, concentrated under vacuum to give crude Bortezomib (19.0 g).

### Purity by HPLC: 96.66%

Isomeric impurity by HPLC: 0.46%
Purification 1: Bortezomib (17 g, Purity: 96.66%) and 5% ethylacetate in Toluene (136 ml) were taken into a round bottom flask and stirred at 25 to 35° C. for 2-3 hours. Filtered the solid obtained under vacuum, washed with 5% ethylacetate in toluene and dried at 50° C. under vacuum for 5 hours to give Bortezomib.

### Yield: 14.0 g (82.3%)

### Purity by HPLC: 98.61%

Isomeric impurity by HPLC: 0.34
Purification 2: Bortezomib (14 g, purity 98.61%) and methanol (42 ml) were taken into a round bottom flask and stirred. Filtered the reaction mass through sintered funnel and washed with methanol (14 ml). Charged filtrate into a round bottom flask and add demineralized water (56 ml) to the reaction mass and stirred for about 2 hours at a temperature of about 27° C. The reaction suspension was filtered and washed the solid with aqueous methanol (84 ml, water:methanol 1:1). The obtained solid was dried at a temperature of about 50° C. for about 5 hours to afford 8.5 g of title compound.

### Yield: 8.5 g (60%)

### Purity by HPLC: 99.84%

Isomeric impurity by HPLC: 0.12%

### HPLC Analysis

### Method-A (for Chemical Purity)

HPLC measurements of Bortezomib samples for chemical purity were performed using Waters system, equipped with Waters symmetry shield RP-18, 250X4.6 mm ID, 5 μm, particle size and a UV detector operated on 270 nm. Analyses were performed using the following mobile phase, at flow rate of 1.0 ml/minute, run time 55 minutes.
Mobile phase A: Mix 700 ml water, 300 ml Acetonitrile and 1 ml formic acid, filter and degas.
Mobile phase B: Mix 800 ml Acetonitrile, 200 ml water and 1 ml formic acid, filter and degas.
Elution: Gradient program

| Time | % A | % B |
|---|---|---|
| 0 | 100 | 0 |
| 15 | 100 | 0 |
| 30 | 0 | 100 |
| 45 | 0 | 100 |
| 47 | 100 | 0 |
| 55 | 100 | 0 |

### Method-B (for Chiral Purity)

HPLC measurements of Bortezomib samples for chiral purity were performed using Waters system, equipped with Amylose tris (3,5 dimethylphenyl carbamate) 250×4.6 (ChiralPak AD-H), coated on 5 μm silica-gel, and a UV detector operated on 270 nm. Analyses were performed using the following mobile phase, at flow rate of 1.0 ml/minute, run time 25 minutes.
Mobile phase: Mix n-Hexane, Isopropyl alcohol and Absolute alcohol in the ratio of 8:1:1.

The stereo isomers and/or impurities of Bortezomib separated in the above methods are characterized by relative retention time (“RRT”). RRT may be calculated by considering the retention time at which the impurity is detected with respect to the retention time of Bortezomib in the HPLC chromatogram. The results of the analysis are depicted in Table 4 and 5.

TABLE 4
Method A (Chemical purity By HPLC)

| | RRT of | | |
|---|---|---|---|
| S.No. | impurities | Example-9 | Example-12 |
| 1 | ~0.36 | N.D. | N.D. |
| 2 | ~0.41 | 0.01 | N.D. |
| 3 | ~0.46 | N.D. | N.D. |
| 4 | ~0.59 | N.D. | N.D. |
| 5 | ~0.88* | N.D. | N.D. |
| 6 | ~1.23** | 0.10 | 0.12 |
| 7 | ~1.51 | N.D. | N.D. |
| 8 | ~1.59 | N.D. | N.D. |
| 9 | ~1.70 | 0.02 | N.D. |
| 10 | ~1.89 | N.D. | 0.02 |
| 11 | ~2.00 | N.D. | 0.01 |
| 12 | ~2.25 | N.D. | 0.01 |
| 13 | ~2.47 | N.D. | N.D. |
| 14 | ~2.70 | N.D. | N.D. |

N.D.: Not detectable
* Represents Impurity-a of the formula:

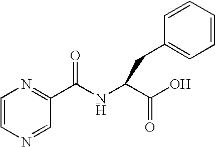

** Represents Impurity-b which is a diastereomeric mixture
of Bortezomib of the comprising compounds of following formulae:
(S,S)-isomer:

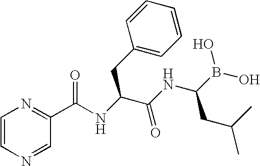

(R,R)-isomer:

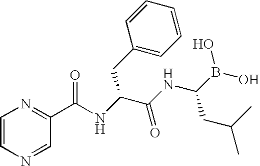

The diastereomeric impurities of Bortezomib along with other isomeric impurities viz,

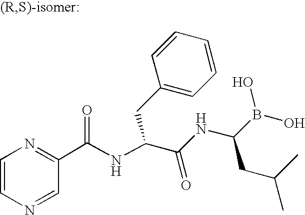

may be separated by Chiral HPLC method (Method B) and their RRT's are summarized in Table-5.

TABLE 5
Method B (Chiral Purity by HPLC)

| | RRT of Chiral | | | |
|---|---|---|---|---|
| S. No. | impurities | Names | Example-5 | Example-12 |
| 1 | ~1.18 | (S,S-isomer) | 0.13% | 0.11% |
| 2 | ~1.35 | (R,S-isomer) | ND | ND |
| 3 | ~1.54 | (R,R-isomer) | 0.03% | 0.02% |
| 4 | ~1.00 | (S,R-Isomer) or | 99.83 | 99.88 |
| | | Bortezomib | | |

1. A process for preparing substantially pure Bortezomib comprising:
a) Providing a solution of Bortezomib in an organic solvent selected from alcohols, halogenated solvents, esters and hydrocarbons, nitriles, hydrocarbons, ethers, or mixtures thereof;
b) adding, where necessary, an anti-solvent to the solution obtained in step a); and
c) isolating the solid product from step a) or step b).

2. The process according to claim 1, wherein said anti-solvent in step b) is selected from water, hydrocarbons, ethers, or mixtures thereof, with the proviso that it is not the same as the solvent used in step a).

3. A process for the preparation of Bortezomib comprising the steps of:
a) reacting (N-[(1S)-2-[[(1R)-1-[(3aS,4S,6S,7aR)-hexahydro-3a,5,5-trimethyl-4,6-methano-1,3,2-benzodioxaborol-2-yl]-3-methylbutylamino]-2-oxo-1-(phenylmethyl)ethyl]Pyrazinecarboxamide (Formula IX) 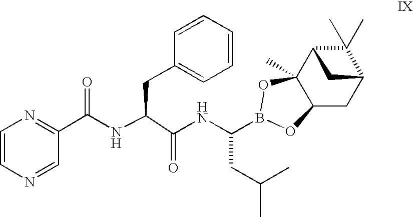
with an organic boronic acid acceptor and aqueous HCl in the presence of an alcohol solvent and an aliphatic hydrocarbon solvent;
b) separating the aqueous layer;
c) extracting the aqueous layer with a water immiscible organic solvent, which is not an aliphatic hydrocarbon solvent; and
d) isolating Bortezomib.

4. The process according to claim 3, wherein said organic boronic acid acceptor is selected from butyl boronic acid, isobutyl boronic acid, phenylboronic acid, and benzyl boronic acid.

5. The process according to claim 3, wherein the amount of organic boronic acid used in step a) ranges from about 1 to about 1.5 molar equivalents, per molar equivalent of the compound of Formula IX.

6. The process according to claim 3, wherein the concentration of aqueous HCl ranges from about 0.5N to about 3N.

7. The process of claim 3, wherein said alcohol solvent is selected from C1-C4 alcohols.

8. The process of claim 3, wherein said hydrocarbon solvent is selected from C4-C10 straight or branched alkanes or cycloalkanes.

9. The process of claim 3, wherein the organic solvent in step c) has solubility in water less than about 10% w/w.

10. The process of claim 3, wherein said water immiscible organic solvent is selected from alcohols (C4-C7), halogenated solvents, esters, or mixtures thereof.

11. The process of claim 3, wherein said isolation in step (d) is performed by cooling, seeding, adding an organic anti-solvent to the reaction mixture or a combination thereof.

12. (canceled)

13. The method of claim 11 wherein said organic anti-solvent is selected from hydrocarbons, halohydrocarbons, esters, or mixtures thereof.

14. A process for the preparation of crystalline Form-A of Bortezomib, which comprises precipitating a crystalline Bortezomib from a solution containing Bortezomib in an alcoholic solvent, wherein said crystalline Bortezomib has an X-ray powder diffraction pattern substantially in accordance with FIG. 2.

15. The process of claim 14, which comprises:
a) providing a solution of Bortezomib in an alcohol;
b) adding water to precipitate the solid; and
c) isolating the crystalline Bortezomib having an X-ray powder diffraction pattern substantially in accordance with FIG. 2.

16. The process of claim 15, wherein said alcohol is methanol or ethanol.

17. A process for the preparation of crystalline Form B of Bortezomib, comprising:
a) providing a solution of Bortezomib in a halogenated alkane solvent or an ester solvent;
b) adding an aromatic hydrocarbon to precipitate a solid; and optionally
c) isolating the obtained solid.

18. The process of claim 17, wherein said crystalline Form B of Bortezomib has an X-ray powder diffraction pattern substantially in accordance with FIG. 5.

19. The process of claim 17, wherein said halogenated alkane is dichloromethane, or 1,2-dichloroethane and chloroform, and said ester solvent is ethyl acetate, isopropyl acetate, tertiary butyl acetate or a mixture thereof.

20. The process of claim 17, wherein said aromatic hydrocarbon solvent in step b) is toluene, xylene or a mixture thereof.

21. A process for the preparation of a compound of Formula III 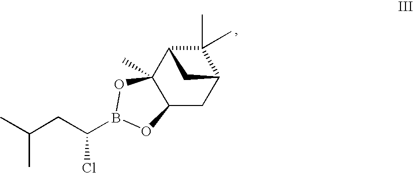
comprising preparing a boronate complex—compound of formula X 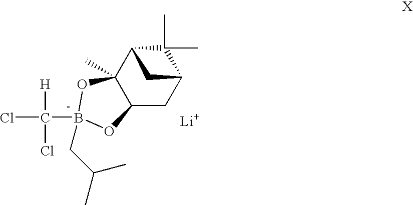
by reacting the compound of formula-II 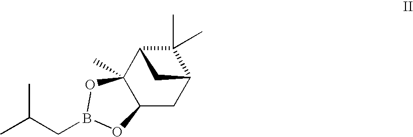
with lithium diisopropyl amide in the presence of a Lewis acid catalyst, a water miscible ether solvent and an excess of dichloromethane, followed by rearrangement of a boronate complex of formula-X.

22. The process of claim 21, wherein dichloromethane is utilized at about 4 Moles to about 8 Moles per mole of compound of formula II.

23. The process of claim 21, wherein a water miscible ether solvent is a cyclic ether solvent.

24. The process of claim 21, wherein a water miscible ether solvent is THF.

25. The process of claim 21, comprising:
I. Adding a lithium diisopropyl amide mixture to a solution of the compound of formula-II 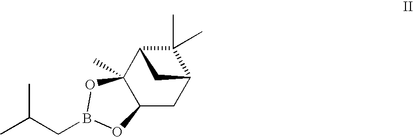
in a solvent mixture comprising dichloromethane and a water miscible ether solvent followed by maintaining the resulting solution at a temperature of about −40 to −70° C.,
II. Adding a mixture of zinc chloride in tetrahydrofuran into the product of step I followed by maintaining the reaction mass at a temperature of about −40 to −70° C.,
III. Raising the reaction temperature to about 10° C. to ambient temperature,
IV. Adding an aqueous acid solution; and
V. Optionally, separating the organic layer containing the compound of formula-III; and
VI. isolating the compound of formula-III.

26. The process of claim 25, wherein said lithium diisopropyl amide mixture is prepared using diisopropyl amine and n-hexyl lithium.

27. The process of claim 25, wherein said organic layer in step V is concentrated to isolate the compound of formula-III.

28. (canceled)

29. The process of claim 25, wherein the amount of Zinc chloride ranges from about 1.2 to about 2 moles per mole of the compound of formula II.

30. The process of claim 21, wherein said Lewis acid catalyst is Zinc chloride.

31. (canceled)

32. A process for the preparation of intermediate of Formula VIII 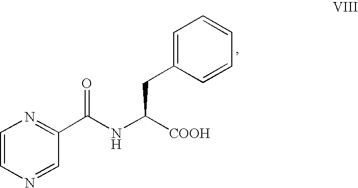
comprising reacting pyrazine carboxylic acid with L-phenylalanine in the presence of an alkyl or aryl chloroformate.

33. The process of claim 32, wherein an alkyl or aryl chloroformate is ethylchloroformate, benzylchloroformate, or para-nitrophenylchloroformate.

34. A compound of the following formula in the solid state.

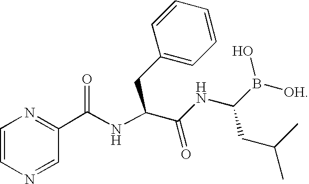

35. The compound according to claim 34 characterized by a peak at m/z=383.19 in negative ion mode in a mass spectral analysis.

36. The compound according to claim 34 characterized by a peak at m/z=367.4 in positive ion mode in a mass spectral analysis.

37. The compound according to claim 34 characterized by an absence of peaks at m/z=1121, 1099, and 1137 in a mass spectral analysis, which peaks correspond to sodium, proton, and potassium adducts of trimeric boroxine.

38. The compound according to claim 34 is, being Bortezomib form A.

39. The compound according to claim 34, having a moisture content up to about 5% w/w.

40. Bortezomib exhibiting a maximum degradation of about 0.2% or from the initial purity when stored for a minimum of about three months.

41. A storage system for Bortezomib comprising:
a. At least one external sealed polymeric bag;
b. At least one internal sealed polymeric bag containing Bortezomib sealed within said at least one external sealed polymeric bag; and
c. An oxygen absorbent and interposed between the external polymeric bag and the internal polymeric bag.

42. The storage system of claim 41 further comprising a moisture absorbent interposed between the external polymeric bag and the internal polymeric bag.

43. The storage system of claim 41 further comprising at least one laminated aluminum bag in which the at least one external sealed polymeric bag is itself sealed.

44. (canceled)

45. The storage system of claim 41 wherein the oxygen absorbent is selected from the group consisting of ascorbic acid, or iron powder containing materials.

46. The storage system of claim 42 wherein the moisture absorbent is selected from the group consisting of aluminum oxide, calcium chloride, CaSO₄, molecular sieves, and silica gel.

47. The storage system according to claim 41 further comprising an inert atmosphere interposed between the external polymeric bag and the internal polymeric bag.
